(19) United States
(12) Patent Application Publication
(10) Pub. No.: US 2017/0347878 A1
(43) Pub. Date: Dec. 7, 2017

(54) A METHOD AND SYSTEM FOR MONITORING AND/OR ASSESSING PUPILLARY RESPONSES

(71) Applicants: SINGAPORE HEALTH SERVICES PTE LTD, Singapore (SG); NATIONAL UNIVERSITY OF SINGAPORE, Singapore (SG)

(72) Inventors: Dan MILEA, Singapore (SG); Joshua James GOOLEY, Singapore (SG)

(21) Appl. No.: 15/536,199

(22) PCT Filed: Dec. 16, 2015
(86) PCT No.: PCT/SG2015/050494

§ 371 Date: Jun. 15, 2017

(30) Foreign Application Priority Data
Dec. 16, 2014 (SG) 10201408408U

(51) Int. Cl. A61B 3/11; A61B 3/14

(52) U.S. Cl. CPC A61B 3/112; A61B 3/14

(57) ABSTRACT

The present invention relates to a method for monitoring and/or assessing pupillary responses to a continuous illumination of light starting at a first intensity which is gradually changed to subsequent intensity over a predefined period of time. The method may be used to detect aberrant pupillary responses. Also included is a system comprising a light source with adjustable light intensity configured to expose one eye of a subject only or each of the eyes of the subject separately, to a continuous illumination of light starting at a first intensity which is gradually changed to a subsequent intensity over a predefined period of time; and a measuring device configured to measure a parameter associated with said eye's pupil response to the change in intensity of the continuous illumination of light at a succession of times over the period of time, to obtain a profile representing the pupillary response of said eye to the change in intensity of the continuous illumination of light corresponding to the succession of times over the predefined period of time.

### FIELD OF THE INVENTION

The present invention relates to a method and system for monitoring and/or assessing the pupillary response of an eye of a subject to a photic stimulus.

### BACKGROUND OF THE INVENTION

Vision is probably the most important sense. Despite the transparency of the anterior part of the eye, allowing visual examination and imaging of the retina and of the optic nerve head, various conditions affecting the visual pathways may be difficult to diagnose, especially at early stages. The concept of early detection or diagnosis is crucial when considering treatment strategies of various ophthalmic conditions affecting the visual pathways, such as diabetic retinopathy or glaucomatous neuropathy, which represent two of the leading causes of blindness, worldwide. As the number of elderly patients in the world rapidly increases, the morbidity related to these ophthalmic conditions will rise, causing increased health care costs and economic burden. Early detection or diagnosis of these conditions is a key factor allowing early treatment and avoidance of irreversible blindness. However, detecting or diagnosing an ophthalmic condition may not be simple, especially in painless conditions, without other patient ophthalmic clinical manifestations. Even visual dysfunction can be quite subtle in various conditions of the eye, leaving the patient unaware of visual loss. For example, in glaucoma, a visual field defect is not detectable prior to a 30% loss of the visual neurons in the retinal ganglion cell layer of the eye. Yet, even then, the patient remains typically asymptomatic, as long as the central part of visual field is still spared, allowing good central vision. This explains why it is currently estimated that between 50-90% of people with glaucoma worldwide are unaware they have the disease, and similar figures may apply to diabetic retinopathy.

Among the numerous ophthalmic conditions, several forms of retinopathies and conditions affecting the optic nerve (including glaucoma) are expected to become the most common causes of irreversible blindness in the world later in the century. Their current detection techniques are not ideal for effective population screening. For example, existing diagnostic approaches for glaucoma such as Humphrey Visual Field (HVF) testing and Heidelberg Retinal Tomography (HRT) are expensive and can only be implemented in the clinic. Moreover, HVF is a subjective assessment, relies heavily on subject responses, is difficult for patients to perform, and is also time-consuming.

In many patients with glaucoma, the progression of the disease may differ for each eye. Hence, damage to retinal ganglion cells (RGCs) and their efferent projections can be detected in these patients based on asymmetric pupillary responses between eyes, referred to as a relative afferent pupillary defect (RAPD). The severity of a RAPD associates with the extent of visual field loss, as well as optic nerve damage. Recently, automated RAPD pupillometry systems have been developed that can potentially be used to screen for patients with glaucoma who exhibit asymmetric disease (Chang et al., 2013; Ozeki et al., (2013) and Tatham et al., (2014). The main limitation of such an approach is that many patients show bilateral damage to the optic nerve without an apparent RAPD. Hence, other complementary approaches are still needed to detect loss of RGC function in patients with symmetric pupillary responses.

Diabetes is another condition which can lead to ocular dysfunction (for example diabetic retinopathy) and eventually blindness if not treated. Early detection of ocular dysfunction in diabetes is of paramount importance to avoid blindness but is problematic since patients do not complain of visual loss until late in the course of the disease. Moreover, the current detection methods based either on clinical examination or on image analysis, are expensive, requiring manpower, appropriate imaging techniques and costly software.

Abnormal pupillary responses have been observed in glaucoma and for other eye diseases affecting optic nerve function and photoreceptor cells in the outer retina (U.S. Pat. No. 7,334,895; Kardon et al., 2010; Kankipati et al., 2011; Ortube et al., 2013) but these studies are of a preliminary nature and, for example, there is limited evidence on whether pupillary responses associate with disease severity.

It is therefore desirable to develop new, objective, affordable and reliable detection methods potentially applicable for a wide range of ocular dysfunctions, conditions and diseases and adaptable for use at a population level, which facilitate early identification of individuals with ocular dysfunctions requiring intervention and/or treatment.

### SUMMARY OF THE INVENTION

According to a first aspect, the present invention provides a method for monitoring and/or assessing a pupillary response of a subject to a photic stimulus comprising:

(a) exposing an eye of the subject to a continuous illumination of light starting at a first intensity and gradually changing the intensity of the light to a subsequent intensity over a predefined period of time;
(b) measuring a parameter associated with said eye's pupil's response to the change in intensity of the continuous illumination of light at a succession of times over the period of time;
(c) thereby obtaining a profile representing the pupillary response of said eye to the change in intensity of the continuous illumination of light corresponding to the succession of times.

The method may be applied to one eye of a subject only, or to each of the eyes of the subject separately. The method may also be applied to one eye only of each subject in a population of subjects, or to each of their eyes separately, thereby obtaining reference profiles representing the pupillary responses of the population.

As a second aspect, the present invention also provides a method for analysing a pupillary response of a subject to a photic stimulus comprising:

(a) exposing an eye of the subject to a continuous illumination of light starting at a first intensity and gradually changing the intensity of the light to a subsequent intensity over a predefined period of time;
(b) measuring a parameter associated with said eye's pupil's response to the change in intensity of the continuous illumination of light at a succession of times over the period of time,
(c) thereby obtaining a profile representing the pupillary response of said eye to the change in intensity of the continuous illumination of light corresponding to the succession of times;
(d) comparing the profile of the subject with reference profiles of at least one population of subjects obtained from performing steps (a), (b), (c) on one eye only or each of the eyes separately of each subject in the population; and
(e) analysing if there is any deviation between the profile of the subject and the reference profiles.

The reference profiles may be from a population of subjects with a medical condition. The reference profiles may also be control reference profiles obtained from a population of control subjects. The profile of the subject may be compared with both the reference profiles from a population of subjects with a medical condition and control reference profiles.

Analyses of the comparison of the profiles may be performed subsequently (for example, performed separately at a later stage), for example, without requiring the presence of the subject.

From the profile comparison, an aberrant pupillary response in a subject or a population of subjects may be detected. For example, a deviation (in particular a significant deviation) between the profile of the subject or profiles of a population of subjects from reference control profiles of a population of control subjects is indicative of an aberrant pupillary response in the subject or population of subjects.

Accordingly, as a third aspect, the present invention provides a method for detecting an aberrant pupillary response associated with a medical condition comprising:

(a) exposing separately one or both of the eyes of a population of subjects with the medical condition to a continuous illumination of light starting at a first intensity and gradually changing the intensity of the light to a subsequent intensity over a predefined period of time;
(b) measuring a parameter associated with said eye's pupil's response to the change in intensity of the continuous illumination of light at a succession of times over the period of time; and
(c) thereby obtaining profiles representing the pupillary responses of the population of subjects with the medical condition to the change in intensity of continuous illumination of light corresponding to the succession of times,
(d) performing steps (a), (b) and (c) on a control population in place of the population of subjects with the medical condition to obtain control reference profiles; and
(e) comparing the profile of the subjects with the medical condition with the control reference profiles; wherein a significant deviation of the profiles of the subjects with the medical condition compared to the control reference profiles is indicative of an aberrant pupillary response associated with the medical condition.

The method according to the third aspect may lead to the development of a test for the medical condition. For example, if a significant deviation suggesting an aberrant pupillary response associated with a medical condition is identified, this may lead to the development of a test for determining if a subject is at risk or suffering from the medical condition.

As a fourth aspect, the present invention provides a method for determining if a subject is at risk of or suffering from a medical condition, comprising:

(a) exposing an eye of the subject to a continuous illumination of light starting at a first intensity and gradually changing the intensity of the light to a subsequent intensity over a predefined period of time;
(b) measuring a parameter associated with said eye's pupil's response to the change in intensity of the continuous illumination of light at a succession of times over the period of time;
(c) thereby obtaining a profile representing the pupillary response to the change in intensity of the continuous illumination of light corresponding to the succession of times; and
(d) comparing the profile of the subject with reference profiles of a population of subjects with the medical condition and control reference profiles of a population of control subjects respectively obtained from performing steps (a), (b), (c) on one eye only or each of the eyes separately of each subject of the population of subjects with the medical condition and the population of control subjects respectively to give fit information; wherein the reference profiles of subjects with the medical condition and the control reference profiles show a significant deviation indicative of an aberrant pupillary response associated with the medical condition;
wherein the subject is determined to be at risk or suffering from the medical condition if the profile of the subject shows a significant fit with the reference profiles of subjects with the medical condition compared with the control reference profiles.

According to a fifth aspect, the present invention provides a method for comparing the pupillary response of a subject before and after administration of a treatment for a medical condition, comprising:

(a) before administration of said treatment, performing steps (i), (ii) and (iii):
(i) exposing an eye of the subject to a continuous illumination of light starting at a first intensity and gradually changing the intensity of the light to a subsequent intensity over a predefined period of time;
(ii) measuring a parameter associated with said eye's pupil's response to the change in intensity of the continuous illumination of light at a succession of times over the period of time;
(iii) thereby obtaining a first profile representing the pupillary response of said eye to the change in intensity of the continuous illumination of light corresponding to the succession of times before administration of said treatment;
   (b) repeating steps (i) and (ii) after administration of said treatment, thereby obtaining a subsequent profile representing the pupillary response of said eye to the change in intensity of the continuous illumination of light corresponding to the succession of times after administration of said treatment;
   (c) comparing the first and subsequent profiles of the subject with each other and analysing for any deviation between the first and subsequent profiles; and/or
   (d) comparing the first and subsequent profiles of the subject with reference profiles of a population of subjects with the medical condition and control reference profiles of a population of control subjects, respectively, to give fit information, said reference and control reference profiles obtained from performing steps (i), (ii), (Hi) on one eye only or each of the eyes separately of each subject of the population of subjects with the medical condition and the population of control subjects, wherein the reference profiles of subjects with the medical condition and the control reference profiles show a significant deviation indicative of an aberrant pupillary response associated with the medical condition.

It will be appreciated that the reference profiles of the population of subjects with the medical condition and the control reference profiles need not be performed de novo and may be retrieved from a tangible computer recordable/readable medium.

According to a sixth aspect, the present invention provides a system for effecting and monitoring a pupillary response to a photic stimulus, the system comprising:

(a) a light source with adjustable light intensity configured to expose, separately, one eye of a subject or each eye independently to a continuous illumination of light starting at a first intensity which is gradually changed to a subsequent intensity over a predefined period of time; and
(b) a measuring device configured to measure a parameter associated with said eye's pupil response to the change in intensity of the continuous illumination of light at a succession of times over the period of time for obtaining a profile representing the pupillary response of said eye to the change in intensity of the continuous illumination of light corresponding to the succession of times over the predefined period of time.

Any suitable measuring device is contemplated. For example, the measuring device may comprise a suitable light source which substantially does not cause any pupillary response (for example an infra-red light source) configured to direct light to the eye, a camera capable of capturing and/or recording images reflected from the eye in real time and a computer processor to take and record measurements of the parameter associated with the pupillary response from each of these images. The camera may be a digital camera.

According to one preferred embodiment of the sixth aspect of the invention, the system comprises:

(a) a light source with adjustable light intensity configured to expose, separately, one eye of a subject or each eye independently to a continuous illumination of light starting at a first intensity which is gradually changed to a subsequent intensity over a predefined period of time;
(b) an infra-red light source configured to direct light to the eye and a camera capable of capturing and/or recording images reflected from the eye in real time;
(c) at least one computer processor configured to carry out computer-executable instructions to control the light intensity, take and record measurements of a parameter associated with the pupillary response of the eye to the change in intensity of the continuous illumination of light from the images, generate a profile from the measurements and analyse the profile; and
(d) at least one tangible computer readable/recordable medium comprising appropriate computer-executable instructions and configured to store further data.

The at least one computer processor in this embodiment may thus generate the profile representing the pupillary response of the eye to the change in intensity of continuous illumination of light corresponding to the succession of times over the predefined time period, and analyse the recorded profiles so as to compare the profile of a subject, reference profiles of subjects with a medical condition and control reference profiles, profiles of a subject before and after administration of a treatment for a medical condition associated an aberrant pupillary response, and determine fit and deviation utilising a suitable algorithm; predict if a subject is at risk or suffering from a medical condition using a suitable algorithm; and/or determine if a subject is responding to a treatment for a medical condition associated with an aberrant pupillary response using a suitable algorithm.

It will be appreciated that this preferred embodiment is for assessing the pupillary response.

According to a seventh aspect, the invention provides a method for determining if a subject is at risk of or suffering from a medical condition comprising:

comparing a profile representing the pupillary response of a subject to a gradual change in intensity of a continuous illumination of light from a first intensity to a subsequent intensity at a succession of times over a predefined time period with:
(i) reference profiles representing separate pupillary responses of one or both eyes of each subject in a population of subjects with the medical condition to the change in intensity of the continuous illumination from the first intensity to the subsequent intensity at a succession of times over a predefined time period; and
(ii) control reference profiles representing separate pupillary responses of one or both eyes of each subject in a population of control subjects to the change in intensity of the continuous illumination of light from the first intensity to the subsequent intensity, respectively, to give fit information;
   wherein the reference profiles of subjects with the medical condition and the control reference profiles show a significant deviation indicative of an aberrant pupillary response associated with the medical condition, and
   wherein the subject is determined to be at risk or suffering from the medical condition if the profile of the subject shows a significant fit with the reference profiles of subjects with the medical condition compared with the control reference profiles.

The present invention may be useful for assessing the effect of treatment for a medical condition in a subject. In particular, an aberrant pupillary response is associated with the medical condition.

According to an eighth aspect, the present invention provides a method for comparing the pupillary response of a subject before and after administration of a treatment for a medical condition, comprising:

comparing a first profile representing the pupillary response of the subject to a gradual change in intensity of a continuous illumination of light from a first intensity to a subsequent intensity at a succession of times over a predefined time period before administration of the treatment with:
a subsequent profile representing the pupillary response of the subject to a gradual change in intensity of a continuous illumination of light from a first intensity to a subsequent intensity at a succession of times over a predefined time period after administration of the treatment; and analysing for any deviation between the first and subsequent profiles;
and/or, comparing the first and subsequent profiles with:
(i) reference profiles representing separate pupillary responses of one or both eyes of each subject in a population of subjects with the medical condition to the change in intensity of the continuous illumination from the first intensity to the subsequent intensity at a succession of times over a predefined time period; and
(ii) control reference profiles representing separate pupillary responses of one or both eyes of each subject in a population of control subjects to the change in intensity of continuous illumination from the first intensity to the subsequent intensity, respectively, to give fit information, wherein the reference profiles of subjects with the medical condition and the control reference profiles show a significant deviation indicative of an aberrant pupillary response associated with the medical condition;
   wherein a significant deviation between the first and subsequent profiles is indicative that the treatment has an effect on the pupillary response; and/or
   wherein the first profile of the subject showing a better fit with the reference profiles of subjects with the medical condition than with the control reference profiles compared to the subsequent profile; and/or
   the subsequent profile of the subject showing a better fit with the control reference profiles than with the reference profiles of subjects with the medical condition compared to the first profile, is indicative of the subject responding positively to the treatment.

It will be appreciated that where an aberrant pupillary response is associated with the medical condition, a significant deviation between the first and subsequent profiles is also indicative that the treatment has an effect on the medical condition.

The present invention may also be useful for assessing and/or monitoring a medical condition associated with an aberrant pupillary response in a subject at different time points. It will be appreciated that the third aspect of the present invention, for example, may be used to determine that an aberrant pupillary response is associated with the medical condition.

According to a ninth aspect, the present invention provides a method for comparing the pupillary response of a subject with a medical condition associated with an aberrant pupillary response at a first time point and a subsequent time point, comprising:

obtaining a first profile representing the pupillary response of the subject to a gradual change in intensity of a continuous illumination of light from a first intensity to a subsequent intensity at a succession of times over a predefined time period at the first time point;
obtaining a subsequent profile representing the pupillary response of the subject to a gradual change in intensity of a continuous illumination of light from a first intensity to a subsequent intensity at a succession of times over a predefined time period at a subsequent time point;
comparing the first and subsequent profiles with control reference profiles representing separate pupillary responses of one or both eyes of each subject in a population of control subjects to the change in intensity of continuous illumination from the first intensity to the subsequent intensity, respectively, to give fit information;
wherein the first profile showing a better fit with the control reference profiles compared with the subsequent profile is indicative that the medical condition in the subject is deteriorating from the first time point to the subsequent time point;
or
wherein the subsequent profile showing a better fit with the control reference profiles compared with the first profile is indicative that the medical condition is improving from the first time point to the subsequent time point.

According to a tenth aspect, the present invention provides a method for comparing the pupillary response of a subject with a medical condition associated with an aberrant pupillary response at a first time point and a subsequent time point, comprising comparing:

a first profile representing the pupillary response of the subject to a gradual change in intensity of a continuous illumination of light from a first intensity to a subsequent intensity at a succession of times over a predefined time period at the first time point, and
a subsequent profile representing the pupillary response of the subject to a gradual change in intensity of a continuous illumination of light from a first intensity to a subsequent intensity at a succession of times over a predefined time period at a subsequent time point; with
control reference profiles representing separate pupillary responses of one or both eyes of each subject in a population of control subjects to the change in intensity of continuous illumination from the first intensity to the subsequent intensity, respectively, to give fit information;
wherein the first profile showing a better fit with the control reference profiles compared with the subsequent profile is indicative that the medical condition in the subject is deteriorating from the first time point to the subsequent time point;
or
wherein the subsequent profile showing a better fit with the control reference profiles compared with the first profile is indicative that the medical condition is improving from the first time point to the subsequent time point.

The present invention may also be useful in comparing pupillary responses at different stages of a medical condition associated with an aberrant pupillary response. It will be appreciated that the third aspect of the present invention, for example, may be used to determine that an aberrant pupillary response is associated with the medical condition.

According to an eleventh aspect, the present invention provides a method of comparing a first population of subjects at a first stage of a medical condition associated with an aberrant pupillary response and a second population of subjects at a more severe stage of the medical condition compared to the first population of subjects, comprising

obtaining a first set of reference profiles representing separate pupillary responses of one or both eyes of each subject in the first population of subjects to a gradual change in intensity of a continuous illumination of light from a first intensity to a subsequent intensity at a succession of times over a predefined time period;
obtaining a second set of reference profiles representing separate pupillary responses of one or both eyes of each subject in the second population of subjects to a gradual change in intensity of a continuous illumination of light from a first intensity to a subsequent intensity at a succession of times over a predefined time period;
comparing the first and second sets of reference profiles with control reference profiles representing separate pupillary responses of one or both eyes of each subject in a population of control subjects to the change in intensity of continuous illumination from the first intensity to the subsequent intensity, respectively, to give fit information;
wherein the first set of reference profiles at a first stage of the medical condition showing less deviation from the control reference profiles compared to the second reference profiles at a more severe stage of the medical condition is indicative that the pupillary response becomes more aberrant with the severity of the condition.

It will be appreciated that the pupillary response of additional populations of subjects at various stages of the medical condition may be compared to identify a correlation between the pupillary response and the severity of the medical condition. Accordingly, the pupillary response may be used to assess the severity of medical condition.

According to a twelfth aspect, the present invention provides a method of comparing a first population of subjects at a first stage of a medical condition associated with an aberrant pupillary response and a second population of subjects at a more severe stage of the medical condition compared to the first population of subjects, comprising comparing:

a first set of reference profiles representing separate pupillary responses of one or both eyes of each subject in the first population of subjects to a gradual change in intensity of a continuous illumination of light from a first intensity to a subsequent intensity at a succession of times over a predefined time period, and
a second set of reference profiles representing separate pupillary responses of one or both eyes of each subject in the second population of subjects to a gradual change in intensity of a continuous illumination of light from a first intensity to a subsequent intensity at a succession of times over a predefined time period; with
control reference profiles representing separate pupillary responses of one or both eyes of each subject in a population of control subjects to the change in intensity of continuous illumination from the first intensity to the subsequent intensity, respectively, to give fit information;
wherein the first set of reference profiles at a first a stage of the medical condition showing less deviation from the control reference profiles compared to the second reference profiles at a more severe stage of the medical condition is indicative that the pupillary response becomes more aberrant with the severity of the condition.

According to a thirteenth aspect, the present invention provides a method of assessing a subject with a medical condition associated with an aberrant pupillary response comprising:

obtaining a profile representing the pupillary response of the subject to a gradual change in intensity of a continuous illumination of light from a first intensity to a subsequent intensity at a succession of times over a predefined time period;
comparing the profile of the subject with a first set of reference profiles representing separate pupillary responses of one or both eyes of each subject in a population of subjects at a first stage of a medical condition, and,
one or more other sets of reference profiles, each other set of reference profile representing separate pupillary responses of one or both eyes of each subject in a population of subjects corresponding to a different stage of the medical condition; and
determining the subject as likely to be at a stage of the medical condition corresponding to the set of reference profiles having the best fit compared to the other reference profiles to the profile of the subject.

According to a fourteenth aspect, the present invention provides a method of assessing a subject with a medical condition associated with an aberrant pupillary response comprising:

comparing a profile representing the pupillary response of the subject to a gradual change in intensity of a continuous illumination of light from a first intensity to a subsequent intensity at a succession of times over a predefined time period with:
a first set of reference profiles representing separate pupillary responses of one or both eyes of each subject in a population of subjects at a first stage of a medical condition, and,
one or more other sets of reference profiles, each other set of reference profile representing separate pupillary responses of one or both eyes of each subject in a population of subjects corresponding to a different stage of the medical condition, and
determining the subject as likely to be at a stage of the medical condition corresponding to the set of reference profiles having the best fit compared to the other reference profiles to the profile of the subject.

In particular, the set of reference profiles having the best fit to the profile of the subject may be regarded as the set of reference profiles having the most significant fit with the profile of the subject compared to the other reference profiles.

It will be appreciated that the appropriate number of other sets of reference profiles, each representing a different stage of the medical condition, depends on the medical condition.

The first stage of the medical condition may be an early stage or mild stage of the medical condition. The other stages of the medical condition would differ from the first stage of the medical condition and from each other in terms of presentations, symptoms, severity. In general, the different stages are stages of the medical condition acknowledged in clinical practice and determined based on typical diagnostic criteria for that stage, and the number of stages of the medical condition depends on the medical condition. The present pupillometry method may help to identify the stage of the medical condition of a subject.

For any or all of the seventh to fourteenth aspects of the invention, the method may be performed on stored profiles, reference profiles of a population of subjects and/or control reference profiles retrieved from a tangible computer recordable/readable medium.

The method according to any aspect of the invention may be performed utilising at least one computer processor. Accordingly, the method according to any aspect of the invention may be implemented using a computer.

### BRIEF DESCRIPTION OF THE FIGURES

FIG. 1. Chromatic pupillometry in control patients with normal-for-age ocular health. (A) Patients were exposed to a 4-minute light exposure sequence consisting of 1 minute of darkness, 2 minutes of monocular exposure to a gradually-increasing blue light (469 nm) or red light (631 nm) stimulus, and 1 minute of darkness after light offset. (B) Representative pupillary constriction responses are shown for a participant who underwent the pupillometry test. (C) The average pupillary response, expressed as a percentage of the dark pupil, is shown across 161 subjects. (D) Dose-response curves are shown for pupillary constriction during exposure to blue light versus red light, with data grouped in 0.5 log unit bins. Asterisks indicate significant differences in percentage pupillary constriction between light conditions. (E) The mean difference in the pupillary constriction response is shown for blue light versus red light, demonstrating greater sensitivity to blue light across a 4 log unit range, from 9 to 13 log photons cm⁻² s⁻¹. In panels C, D, and E, the mean±SEM is shown.

FIG. 2. Chromatic pupillometry in patients with glaucoma. Representative pupillary constriction responses are shown for patients with (A) mild stage glaucoma, (B) moderate stage glaucoma, or (C) severe stage glaucoma. The pupillary constriction response was reduced in patients with greater disease severity. As in FIG. 1, patients were exposed to 1 minute of darkness, 2 minutes of monocular exposure to a gradually-increasing blue light (469 nm) or red light (631 nm) stimulus, and 1 minute of darkness. (D) Dose-response curves are shown for pupillary constriction during exposure to blue light versus red light, assessed in 40 patients with glaucoma. Asterisks indicate significant differences in the response between light conditions. (E) Patients exhibited greater sensitivity to blue light in the range from 9 to 13 log photons cm⁻² s⁻¹, based on the mean difference in the pupillary constriction for blue light versus red light. In panels D and E, the mean±SEM is shown.

FIG. 3. Impaired pupillary constriction responses in patients with glaucoma. (A) Dose-response curves for pupillary constriction are shown for controls (n=161, black traces) and patients with glaucoma (n=40) who were exposed to blue 469 nm light (blue trace), and (B) red 631 nm light (red trace). For both colours of light, the magnitude of the pupillary light reflex (PLR) was reduced in glaucomatous eyes as the irradiance of light was increased (>11.5 log photons cm⁻² s⁻¹). Asterisks show significant differences in pupillary responses between controls and patients with glaucoma. The mean±SEM is shown. (C) In patients with glaucoma, the pupillary constriction response to high-irradiance blue light (13 to 14 log photons cm⁻² s⁻¹) correlated significantly with visual field deficits assessed by Humphrey Visual Field (HVF) testing, as well as (D) the linear cup disc ratio determined by Heidelberg retinal tomography. The linear regression line is shown with 95% Confidence Intervals.

FIG. 4. Pupillary constriction responses correlated with clinical measures used to diagnose glaucoma. The heat maps show Pearson's correlation coefficient for visual field testing and optic nerve head parameters versus pupillary constriction in a group of 40 patients with glaucoma. Correlations with pupillary responses to blue light (469 nm) and red light (631 nm) are shown in 0.5 log unit bins from 7 to 14 log photons cm⁻² s⁻¹. Hotter colours indicate higher correlation coefficient values. Results for Humphrey Visual Field (HVF) analysis and Heidelberg retinal tomography correlated most strongly with the magnitude of pupillary constriction during exposure to high-irradiance blue light.

FIG. 5. Impaired pupillary constriction associated with glaucoma disease severity. Dose-response curves for pupillary constriction are shown for patients with early glaucoma (n=29), moderate glaucoma (n=14), and severe glaucoma (n=12) who were exposed to (A) blue 469 nm light (blue trace), and (B) red 631 nm light (red trace). Asterisks show significant differences in pupillary responses between controls and patients with glaucoma. The mean±SEM is shown.

FIG. 6. Impaired pupillary constriction responses in patients with normal tension glaucoma (NTG). Dose-response curves for pupillary constriction are shown for NTG patients (n=26) who were exposed to (A) blue 469 nm light (blue trace), and (B) red 631 nm light (red trace). Asterisks show significant differences in pupillary responses between controls and patients with glaucoma. The mean±SEM is shown.

FIG. 7. Impaired pupillary constriction responses in patients who were primary angle closure suspects (PACS). Dose-response curves for pupillary constriction are shown for PACS patients (n=14) who were exposed to (A) blue 469 nm light (blue trace), and (B) red 631 nm light (red trace). Asterisks show significant differences in pupillary responses between controls and patients with glaucoma. The mean±SEM is shown.

FIG. 8. Chromatic pupillometry results in a patient with severe, unilateral optic neuritis. (A) During an attack of optic neuritis, there was profound visual field loss in the left eye, and (B) no detectable pupillary response during exposure to blue 469 nm light or red 631 nm light. Coloured traces show the patient's pupil diameter, as compared to a group of 20 controls aged 63 to 67 years (black traces±grey lines=mean±SD) with normal ophthalmic examination, who underwent the same testing procedures. The ramp-up light stimulus is shown at the top of each plot. (C) Recovery of vision and of the visual field was associated with (D) normal pupillary light responses relative to age-matched controls.

FIG. 9. Impaired pupillary constriction responses in patients with diabetic retinopathy. A patient with diabetic retinopathy exhibited blunted pupillary responses to a gradually increasing (A) blue 469 nm light and (B) red 631 nm light. A second patient with diabetic retinopathy showed impaired pupillary constriction in response to (C) blue light and (D) red light. For comparison, pupillary constriction responses in age-matched controls are shown by the coloured traces with standard deviation indicated in grey.

FIG. 10. Impaired pupillary constriction response in patient with retinitis pigmentosa (RP). As compared to control patients of similar age who were exposed to a gradually increase 469 nm blue light or 631 nm red light (left panel), the RP patient showed no pupillary constriction to red light, and a delayed pupillary constriction response to blue light.

FIG. 11 shows an embodiment of the desktop system; (A) shows a pictorial depiction of the desktop system, (B) shows a cut-out view, (C) shows a cross-sectional view along the line AA of (B).

FIG. 12 shows an embodiment of a wearable system in the form of a pair of goggles; A) shows a pictorial depiction of the desktop system, (B) shows a cut-out view, (C) shows a cross-sectional view along the line BB of (A).

FIG. 13 shows a plan illustrating components of the system. The components are: 1. Multiple LED sources of any wavelength with computer control of brightness; 2. Mixer and diffuser that combine light from LED's to produce uniform field of illumination; 3. Relay optics and pattern masks that allow light to be directed toward the eye—a visible fixation target can also be added at this point; 4. Beam splitter that transmits visible light to patient and directs returned IR light to camera; 5. Relay optics that transmit and receive light to and from the eye, and provide comfortable working distance to patient; 6. Patient's Eye—illuminated by light from visible LED's and IR source for camera; 7. Camera and IR illumination system that detect pupil diameter changes in patient's eye and 8. Computer that controls LED brightness and analyses the pupil diameter during examination.

### DEFINITIONS

The term “narrow bandwidth” is used to refer to light sources that emit light within a narrow band of wavelengths; for example, the width of the band of light is typically 20-50 nm at one-half the peak maximum. The peak wavelength as well as the two wavelength values at one-half the peak maximum are used to define the light emitted by a narrow bandwidth illuminator. Exemplary typical light sources having a narrow bandwidth characteristic are red, blue, and green LEDs (light-emitting diodes). Narrow bandwidth light sources, as referred to in the specification and claims herein below, typically exhibit light in wavelengths substantially in the visible spectrum.

A subject with a medical condition is diagnosed with the medical condition based on the typical diagnostic criteria for that medical condition.

A “control subject” or a “population of control subjects” is taken to mean a typically healthy individual or a population of such healthy individuals. In particular, the control subject or population of control subjects is not suffering from a specific medical condition with which a subject is to be compared.

### DETAILED DESCRIPTION OF THE INVENTION

According to a first aspect, the present invention provides a method for monitoring and/or assessing a pupillary response of a subject to a photic stimulus comprising:

(a) exposing an eye of the subject to a continuous illumination of light starting at a first intensity and gradually changing the intensity of the light to a second intensity over a predefined period of time;
(b) measuring a parameter associated with said eye's pupil's response to the change in intensity of the continuous illumination of light at a succession of times over the period of time;
(c) thereby obtaining a profile representing the pupillary response of said eye to the change in intensity of the continuous illumination of light corresponding to the succession of times.

The method may be applied to one eye of a subject only, or to each of the eyes of a subject separately. The method may also be applied to one eye only of each subject in a population of subjects, or to each of their eyes separately, thereby obtaining reference profiles representing the pupillary responses of the population.

As a second aspect, the present invention also provides a method for analysing a pupillary response of a subject to a photic stimulus comprising:

(a) exposing an eye of the subject to a continuous illumination of light starting at a first intensity and gradually changing the intensity of the light to a subsequent intensity over a predefined period of time;
(b) measuring a parameter associated with said eye's pupil's response to the change in intensity of the continuous illumination of light at a succession of times over the period of time,
(c) thereby obtaining a profile representing the pupillary response of said eye to the change in intensity of the continuous illumination of light corresponding to the succession of times;
(d) comparing the profile of the subject with reference profiles of at least one population of subjects obtained from performing steps (a), (b), (c) on one eye only or each of the eyes separately of each subject in the population; and
(e) analysing if there is any deviation between the profile of the subject and the reference profiles.

The reference profiles may be from a population of subjects with a medical condition. The reference profiles may also control reference profiles obtained from a population of control subjects. The profile of the subject may be compared with both the profiles from a population of subjects with a medical condition and control reference profiles.

Analyses of the comparison of the profiles may be performed subsequently (for example, performed separately at a later stage), for example, without requiring the presence of the subject.

From the profile comparison, an aberrant pupillary response in a subject or a population of subjects may be detected. For example, a deviation (in particular a significant deviation) between the profile of the subject or profiles of a population of subjects from reference control profiles of a population of control subjects is indicative of an aberrant pupillary response in the subject or population of subjects.

A comparison between the reference profiles of a population of subjects with a medical condition and the control reference profiles may also be performed. A significant deviation between the reference profiles from a population of subjects with a medical condition and the control reference profiles is indicative of an aberrant pupillary response associated with the medical condition.

Subsequent analyses may also determine if a subject is at risk of or suffering from such a medical condition. For example, in subsequent analyses of the profile of the subject, the profile of the subject is compared with the reference profiles of a population of subjects with a medical condition and control reference profiles to ascertain the extent to which the subject's profile fits with the profiles of either reference group; wherein the subject is determined to be at risk or suffering from the medical condition if the profile of the subject shows a significant fit with the reference profiles of subjects with the medical condition compared with the control reference profiles.

The method may also be used to detect if the medical condition is improving or worsening. Subsequent profiles of the pupillary responses of the subject may be similarly obtained and compared with earlier profiles of the same subject, as well as with the reference profiles of a population of subjects with the medical condition and control reference profiles. A subsequent profile of the subject showing a better fit with the control reference profiles compared with the reference profiles of a population of subjects with the medical condition than a prior profile of the subject is indicative that the medical condition is improving. On the other hand, a subsequent profile of the subject showing a better fit with the reference profiles of a population of subjects with the medical condition than the control reference profiles is indicative that the medical condition is worsening.

Accordingly, as a third aspect, the present invention provides a method for detecting an aberrant pupillary response associated with a medical condition comprising:

(a) exposing separately one or both of the eyes of a population of subjects with the medical condition to a continuous illumination of light starting at a first intensity and gradually changing the intensity of the light to a subsequent intensity over a predefined period of time;
(b) measuring a parameter associated with said eye's pupil's response to the change in intensity of the continuous illumination of light at a succession of times over the period of time; and
(c) thereby obtaining profiles representing the pupillary responses of the population of subjects with the medical condition to the change in intensity of the continuous illumination of light corresponding to the succession of times,
(d) performing steps (a), (b) and (c) on a control population in place of the population of subjects with the medical condition to obtain control reference profiles; and
(e) comparing the profile of the subjects with the medical condition with the control reference profile; wherein a significant deviation of the profiles of the subjects with the medical condition compared to the control reference profiles is indicative of an aberrant pupillary response associated with the medical condition.

An aberrant pupillary response may be due to a defect in rods, cones and/or melanopsin-containing photosensitive cells. An example of melanopsin-containing photosensitive cells are melanopsin-containing retinal ganglion cells.

The medical condition referred to in any relevant aspect of the invention includes but is not limited to glaucoma (e.g. open angle glaucoma, angle closure glaucoma or normal tension glaucoma), diabetes (in particular diabetic retinopathy), damage to the outer retina (e.g. retinitis pigmentosa), macular dystrophy, age-related macular degeneration and optic neuritis. It will be appreciated that the method of the present invention may also be applied to other visual abnormalities.

The medical condition may also include but is not limited to a neurological condition such as dementia or an extrapyramidal syndrome (e.g. Parkinson's disease) and an inflammatory condition (e.g. multiple sclerosis or neuromyelitis optica). It will be appreciated that a neurological condition include a neurodengenerative condition (e.g. Alzheimer's disease). It will be appreciated that extrapyramidal syndromes include other motor disorders or movement disorders,

For the purposes of the present invention, the intensity of the light is gradually changed from a first intensity to a subsequent intensity.

The rate of change in intensity may be constant or not constant. The rate of change of intensity may be logarithmic. In particular, the rate of change in intensity may be described by the number of steps taken to reach the subsequent intensity from the first intensity over a predefined time period. For example, a gradual change in intensity of the light may be increasing the intensity from a first intensity of 11.5 log photons cm² s⁻¹ in 1400 steps to a subsequent intensity of 14.5 log photons cm² s⁻¹ over a predefined time period of 2 minutes.

Different first, second intensity and/or number of steps of change in intensity may be suitable or relevant for different medical conditions, or for assessing or monitoring different ocular abnormalities. The present invention is not intended to be limited to a particular first intensity, second intensity, number of steps of change in intensity, or the predefined time period of exposure to the light.

For example, the first and second intensities may each be any intensity ranging from 0 to 18 log photons cm⁻² s⁻¹,

Any suitable predefined time period of exposure to the continuous illumination of light may be applied. This predefined time period should ideally not be too long so as not to bother or inconvenience the subject but still provide a meaningful profile. For example, this predefined time period includes but is not limited to any time period selected from 0.5 to 5 minutes.

It will be appreciated that the number of steps applied to change from the first intensity to a subsequent intensity may also be varied as appropriate.

The subsequent intensity of the light may be higher than the first intensity; in which case, the intensity of the light is increased from the first intensity to the subsequent intensity. Alternatively, the subsequent intensity of light may be lower than the first intensity, in which case the intensity of the light is decreased from the first intensity to the subsequent intensity.

The intensity of the light may be gradually changed from a first intensity to a second intensity to a third intensity to a fourth intensity etc. It will be appreciated that the intensity may increase or decrease when changed from the first intensity to the second intensity independently of each subsequent change in intensity which may also either increase or decrease independently.

The method according to the third aspect may lead to the development of a test for the medical condition. For example, if a significant deviation suggesting an aberrant pupillary response associated with a medical condition is identified, this may lead to the development of a test for determining if a subject is at risk or suffering from the medical condition. Different wavelengths may be tested to determine the most suitable wavelength or narrow bandwidth wavelength which gives a significant deviation. Similarly, different first intensity, subsequent intensity, and rates of intensity changes and the predefined time period of exposure to the light may be tested to detect a significant deviation between profiles of a population of subjects with the medical condition and control reference profiles.

In one embodiment, glaucoma patients exhibited impaired pupillary responses to blue light when the intensity (irradiance) was increased beyond 11.5 log photons cm⁻² s⁻¹. The impaired pupillary responses to high-intensity (high-irradiance) blue light (e.g. beyond 11.5 log photons cm⁻² s⁻¹) is associated with, for example, loss of visual field, which is indicative of the severity of glaucoma.

As a fourth aspect, the present invention provides a method for determining if a subject is at risk of or suffering from a medical condition, comprising:

(a) exposing an eye of the subject to a continuous illumination of light starting at a first intensity and gradually changing the intensity of the light to a subsequent intensity over a predefined period of time;
(b) measuring a parameter associated with said eye's pupil's response to the change in intensity of the continuous illumination of light at a succession of times over the period of time;
(c) thereby obtaining a profile representing the pupillary response to the change in intensity of the continuous illumination of light corresponding to the succession of times; and
(d) comparing the profile of the subject with reference profiles of a population of subjects with the medical condition and control reference profiles of a population of control subjects respectively obtained from performing steps (a), (b), (c) on one eye only or each of the eyes separately of each subject of the population of subjects with the medical condition and the population of control subjects respectively to give fit information; wherein the reference profiles of subjects with the medical condition and the control reference profiles show a significant deviation indicative of an aberrant pupillary response associated with the medical condition;
wherein the subject is determined to be at risk or suffering from the medical condition if the profile of the subject shows a significant fit with the reference profiles of subjects with the medical condition compared with the control reference profiles.

It will be appreciated that the method according to the fourth aspect of the invention may be applied in combination with other methods such as post-illumination pupillary response, and a method to detect relative afferent pupillary defect (RAPD) to enhance accuracy and/or sensitivity.

According to a fifth aspect, the present invention provides a method for comparing the pupillary response of a subject before and after administration of a treatment for a medical condition, comprising:

(a) before administration of said treatment, performing steps (i), (ii) and (iii):
(i) exposing an eye of the subject to a continuous illumination of light starting at a first intensity and gradually changing the intensity of the light to a subsequent intensity over a predefined period of time;
(ii) measuring a parameter associated with said eye's pupil's response to the change in intensity of the continuous illumination of light at a succession of times over the period of time;
(iii) thereby obtaining a first profile representing the pupillary response of said eye to the change in intensity of the continuous illumination of light corresponding to the succession of times before administration of said treatment;
   (b) repeating steps (i) and (ii) after administration of said treatment, thereby obtaining a subsequent profile representing the pupillary response of said eye to the change in intensity of the continuous illumination of light corresponding to the succession of times before administration of said treatment;
   (c) comparing the first and subsequent profiles of the subject with each other and analysing for any deviation between the first and subsequent profiles; and/or
   (d) comparing the first and subsequent profiles of the subject with reference profiles of a population of subjects with the medical condition and control reference profiles of a population of control subjects, respectively, to give fit information, said reference and control reference profiles obtained from performing steps (i), (ii), (iii) on one eye only or each of the eyes separately of each subject of the population of subjects with the medical condition and the population of control subjects, wherein the reference profiles of subjects with the medical condition and the control reference profiles show a significant deviation indicative of an aberrant pupillary response associated with the medical condition.

It will be appreciated that the reference profiles of the population of subjects with the medical condition and the control reference profiles need not be performed de novo and may be retrieved from a tangible computer recordable/readable medium.

Further analyses of the profiles before and after administration of treatment may be performed subsequently (for example, performed separately at a later stage). For example, on subsequent analyses, a deviation (in particular a significant deviation) between the first and subsequent profiles suggests that the treatment has an effect on the pupillary response.

On subsequent analyses, the first profile is compared and fitted with the reference profiles of the subjects with the medical condition and the control reference profiles. The subsequent profile is similarly compared and fitted with the profiles of the subjects with the medical condition and the control reference profiles. The first profile showing a better fit with the reference profiles of the population of subjects with the medical condition than the reference profiles of the population of control subjects compared with the subsequent profile and/or the subsequent profile showing a better fit with the reference profiles of the population of control subjects than the reference profiles of the population with the medical condition compared to the first profile is indicative that the subject is responding positively to the treatment.

It will be appreciated that where an aberrant pupillary response is associated with the medical condition, a significant deviation between the first and subsequent profiles is also indicative that the treatment has an effect on the medical condition.

It will be appreciated that if the treatment comprises a protocol of several rounds of administration, subsequent profiles of the pupillary response of the subject may be generated after each round of administration. It will further be appreciated that the first profile includes any profile generated before any round of administration and the subsequent profile includes any profile generated after any round of administration. The subsequent profiles may be compared with each other and with any profile before administration of any treatment to assess the response of the subject with each round of administration of the treatment.

The method according to any one of the first to fifth aspects of the invention may be performed on one eye of a subject only, or on each of the eyes of the subject separately, or on one eye only of each of a population of subjects, or on each of their eyes separately.

It will be appreciated that the method according to any one of the first to fifth aspects of the invention may be performed using a system comprising a light source with adjustable light intensity configured to expose one eye of a subject only, or each of the eyes of the subject separately, to a continuous illumination of light starting at a first intensity which is gradually changed to a subsequent intensity over a predefined period of time, and to a measuring device configured to measure a parameter associated with said eye's pupil response.

Pupillometric measurements of the eye of the subject under dark-adapted conditions may additionally be performed. These measurements are taken when the eye is in a substantially darkened environment and is not exposed to visible light or any light capable of causing a pupillometric response for any suitable period of time. It will be appreciated that these measurements need not be in complete darkness and may also be taken in for example, a dim environment. These pupillometric measurements will be considered baseline measurements of the parameter and may be compared with the changes in the parameter of the pupil on exposure to the light.

According to a sixth aspect, the present invention provides a system for effecting and monitoring a pupillary response to a photic stimulus, the system comprising:

(a) a light source with adjustable light intensity configured to expose, separately, one eye only of a subject or each of the eyes independently to continuous illumination starting at a first intensity which is gradually changed to a subsequent intensity over a predefined period of time; and
(b) a measuring device configured to measure a parameter associated with said eye's pupil response to the change in intensity of the continuous illumination of light at a succession of times over the period of time for obtaining a profile representing the pupillary response of said eye to the change in intensity of the continuous illumination of light corresponding to the succession of times over the predefined period of time.

It will be appreciated that the system will require a suitable power source.

Any suitable measuring device is contemplated. For example, the measuring device may comprise a suitable light source which substantially does not cause any pupillary response (for example an infra-red light source) configured to direct light to the eye, camera capable of capturing and/or recording images reflected from the eye in real time and a computer processor to take and record measurements of the parameter associated with the pupillary response from each of these images. The camera may be a digital camera.

In another example, the measuring device scans the eye, takes and records measurements of the parameter associated with the pupillary response directly in real time.

As an alternative, the system may comprise:

(a) a light source with adjustable light intensity configured to expose, separately, one eye of a subject or each eye independently to a continuous illumination of light starting at a first intensity which is gradually changed to a subsequent intensity over a predefined period of time; and
(b) a suitable light source which substantially does not cause any pupillary response (for example an infra-red light source) configured to direct light to the eye and a camera capable of capturing and/or recording images reflected from the eye in real time.

The real-time images may then subsequently be transferred to a second computer processor which takes and records measurements of the parameter associated with the pupillary response from each of the images. This second computer processor is an independent unit from the system of the invention but may be connected to the system via a server.

The system is configured with a light source producing the desired light. The light source may be a monochromatic light source or a light source of a narrow bandwidth wavelength. The system may comprise a light emitting diode as the light source. Alternatively, the system may comprise a laser as the light source. The system may include light filters to produce the monochromatic or narrow-bandwidth light from a light source that provides broadband light or, alternatively, white light. It will be appreciated that the light source in the system may be changed to produce the desired light, as appropriate. It will be appreciated that two or more types of light sources (of different wavelengths) may be applied separately or in combination to the same subject.

The continuous illumination of light may be directed to substantially the entire retina of the eye. Alternatively, the continuous illumination of light may be directed to a selected target region of the retina of the eye. For example, this may be achieved via filters or mirrors or other reflective surface which direct the continuous illumination of light to the selected target region (e.g. inferior, superior, nasal, temporal) of the retina of the eye. In practice, the subject is typically required to fixate on a stationary visual target while the light is directed to the selected target region. In one embodiment, the target region may be circular; for example, a circular central region of the retina (that is, a filter blocks light to an annular peripheral region of the retina). In another embodiment, the target region may be annular; for example, an annular peripheral region of the retina (that is, a filter blocks light to a circular central region of the retina). In the situation where a mirror or other reflective surface is used, the light may be directed to different parts of the retina, e.g. different quadrants of the retina.

Any suitable light may be applicable. The light may be a visible light. The light may be polychromatic or monochromatic. In particular, the light may be of a narrow bandwidth wavelength. The light may be from a light emitting diode. More in particular, the light may be blue light or red light. For example, the light may be blue light of a narrow bandwidth wavelength with a peak at 469 nm or red light of a narrow bandwidth with a peak at 631 nm. The light may also be monochromatic blue light with wavelength of 469 nm or monochromatic red light with wavelength of 631 nm. The light may also include a combination comprising two or more lights of different wavelengths, each selected independently from the group consisting of a monochromatic light and a narrow bandwidth wavelength light. For example, the combination may comprise two or more monochromatic lights, two or more narrow bandwidth lights, any combination of monochromatic light and narrow bandwidth light. The light may also be white light.

It will be appreciated that the light stimulus may comprise one or more sequences of continuous illumination of light starting at a first intensity and gradually changing the intensity of the light to a subsequent intensity over a predefined period of time. Each sequence may differ from another sequence in terms of wavelength, chromaticity, duration, the intensity range and the rate of change of intensity. Each light sequence may begin immediately after another one or be separated by a period of darkness or constant light intensity. For example, a first stimulus sequence may comprise a continuous blue light with a gradually changing intensity and a subsequent stimulus sequence may comprise a continuous red light with a gradually changing intensity; or vice versa. It will be appreciated that any number of sequences may be applied. In another example, the stimulus sequence can first be a red light at low intensities followed by a blue light at higher intensities.

Each sequence may vary from another sequence in terms of at least one of wavelength, chromaticity, duration, the intensity range and the rate of change of intensity. A sequence sharing at least one of wavelength, chromaticity, duration the intensity range and the rate of change of intensity with a sequence already used before may be repeated again. By using more than one sequence of continuous light stimulation, which may vary in wavelengths and intensities and durations, the present invention may be applied to more ocular abnormalities, by tailoring the stimulus accordingly to discriminate the responses of the visual photoreceptors and ipRGCs.

Accordingly, the method comprises exposing the eye to multiple sequences of a continuous illumination of light starting at a first intensity and gradually changing the intensity of the light to a subsequent intensity over a predefined period of time.

Accordingly, the system may be adapted to present multiple sequences of a continuous illumination of light starting at a first intensity and gradually changing the intensity of the light to a subsequent intensity over a predefined period of time.

The system also comprises a computer processor. The computer processor is configured to control the light intensity; control the filters to direct the light source to selected target region of the retina and the light filters to produce the monochromatic light where applicable, control the measuring device, obtain the profile representing the pupillary response of said eye to the change in intensity of continuous illumination corresponding to the succession of times over the predefined period of time, compare and analyse the profile of a subject, reference profiles of subjects with a medical condition and control reference profiles, profiles of a subject before and after administration of a treatment for a medical condition associated an aberrant pupillary response, determine fit and deviation utilising a suitable algorithm, determine normal or aberrant pupillary response using a suitable algorithm, predict if a subject is at risk or suffering from a medical condition using a suitable algorithm, and/or determine if a subject is responding to a treatment for a medical condition associated with an aberrant pupillary response using a suitable algorithm, as applicable.

The system may also comprises a computer readable/recordable medium configured to store data.

According to one preferred embodiment of the sixth aspect of the invention, the system comprises:

(a) a light source with adjustable light intensity configured to expose, separately, one eye of a subject or each eye independently to a continuous illumination of light starting at a first intensity which is gradually changed to a subsequent intensity over a predefined period of time;
(b) an infra-red light source configured to direct light to the eye and a camera capable of capturing and/or recording images reflected from the eye in real time;
(c) at least one computer processor configured to carry out computer-executable instructions to control the light intensity, take and record measurements of a parameter associated with the pupillary response of the eye to the change in intensity of the continuous illumination of light from the images, generate a profile from the measurements and analyse the profile; and
(d) at least one tangible computer readable/recordable medium comprising appropriate computer-executable instructions and configured to store further data.

The at least one computer processor in this embodiment may thus generates the profile representing the pupillary response of the eye to the change in intensity of continuous illumination of light corresponding to the succession of times over the predefined time period, and analyses the recorded profiles so as to compare the profile of a subject, reference profiles of subjects with a medical condition and control reference profiles, profiles of a subject before and after administration of a treatment for a medical condition associated an aberrant pupillary response, and determine fit and deviation utilising a suitable algorithm; predict if a subject is at risk or suffering from a medical condition using a suitable algorithm; and/or determine if a subject is responding to a treatment for a medical condition associated with an aberrant pupillary response using a suitable algorithm.

It will be appreciated that this preferred embodiment is for assessing the pupillary response.

It will be appreciated that the system may comprise a display interface to display data of the profiles, analyses, comparisons, whether the subject is at risk or suffering from a medical condition and/or how the subject is responding to treatment.

The system according to this embodiment may be in the form of a desktop system or a wearable device.

It will be appreciated that the images, measured parameters, the profiles, reference profiles, control reference profiles, fit information, whether an aberrant pupillary response is associated with a medical condition, information as to whether a subject is at risk or suffering from a medical condition, information as to whether a subject is responding to a treatment for a medical condition associated with an aberrant pupillary response, information as to pupillary responses at different stages of a medical condition and/or information as to the stage of a medical condition in a subject based on the pupillary response of the subject, as applicable, obtained from a method according to any aspect of the invention may be stored as data in a tangible computer recordable/readable medium.

Accordingly, the system of the present invention may also comprise a tangible computer recordable/readable medium configured to store data.

The system may be a portable system. The system may be a desktop system. It will be appreciated that the desktop system may also be portable. The system may also be incorporated into a wearable device. For example, the system is incorporated into a pair of goggles to be worn by the subject.

According to a seventh aspect, the invention provides a method for determining if a subject is at risk of or suffering from a medical condition comprising:

comparing a profile representing the pupillary response of a subject to a gradual change in intensity of a continuous illumination of light from a first intensity to a second intensity at a succession of times over a predefined time period with:
(i) reference profiles representing separate pupillary responses of one or both eyes of each subject in a population of subjects with the medical condition to the change in intensity of the continuous illumination from the first intensity to the subsequent intensity at a succession of times over a predefined time period; and
(ii) control reference profiles representing separate pupillary responses of one or both eyes of each subject in a population of control subjects to the change in intensity of the continuous illumination of light from the first intensity to the subsequent intensity, respectively, to give fit information;
   wherein the reference profiles of subjects with the medical condition and the control reference profiles show a significant deviation indicative of an aberrant pupillary response associated with the medical condition, and
   wherein the subject is determined to be at risk or suffering from the medical condition if the profile of the subject shows a significant fit with the reference profiles of subjects with the medical condition compared with the control reference profiles.

The present invention may be useful for assessing the effect of treatment for a medical condition in a subject. In particular, an aberrant pupillary response is associated with the medical condition.

According to an eighth aspect, the present invention provides a method for comparing the pupillary response of a subject before and after administration of a treatment for a medical condition, comprising:

comparing a first profile representing the pupillary response of the subject to a gradual change in intensity of a continuous illumination of light from a first intensity to a subsequent intensity at a succession of times over a predefined time period before administration of the treatment with:
a subsequent profile representing the pupillary response of the subject to a gradual change in intensity of a continuous illumination of light from a first intensity to a subsequent intensity at a succession of times over a predefined time period after administration of the treatment; and analysing for any deviation between the first and subsequent profiles;
and/or, comparing the first and subsequent profiles with:
(i) reference profiles representing separate pupillary responses of one or both eyes of each subject in a population of subjects with the medical condition to the change in intensity of the continuous illumination from the first intensity to the subsequent intensity at a succession of times over a predefined time period;
and
(ii) control reference profiles representing separate pupillary responses of one or both eyes of each subject in a population of control subjects to the change in intensity of the continuous illumination of light from the first intensity to the subsequent intensity, respectively, to give fit information, wherein the reference profiles of subjects with the medical condition and the control reference profiles show a significant deviation indicative of an aberrant pupillary response associated with the medical condition;
wherein a significant deviation between the first and subsequent profiles is indicative that the treatment has an effect on the pupillary response; and/or
wherein the first profile of the subject showing a better fit with the reference profiles of subjects with the medical condition than with the control reference profiles compared to the subsequent profile; and/or
the subsequent profile of the subject showing a better fit with the control reference profiles than with the reference profiles of subjects with the medical condition compared to the first profile; is indicative of the subject responding positively to the treatment.

It will be appreciated that where an aberrant pupillary response is associated with the medical condition, a significant deviation between the first and subsequent profiles is also indicative that the treatment has an effect on the medical condition.

The present invention may also be useful for assessing and/or monitoring a medical condition associated with an aberrant pupillary response in a subject at different time points. It will be appreciated that the third aspect of the present invention, for example, may be used to determine that an aberrant pupillary response is associated with the medical condition.

According to a ninth aspect, the present invention provides a method for comparing the pupillary response of a subject with a medical condition associated with an aberrant pupillary response at a first time point and a subsequent time point, comprising:

obtaining a first profile representing the pupillary response of the subject to a gradual change in intensity of a continuous illumination of light from a first intensity to a subsequent intensity at a succession of times over a predefined time period at the first time point;
obtaining a subsequent profile representing the pupillary response of the subject to a gradual change in intensity of a continuous illumination of light from a first intensity to a subsequent intensity at a succession of times over a predefined time period at a subsequent time point;
comparing the first and subsequent profiles with control reference profiles representing separate pupillary responses of one or both eyes of each subject in a population of control subjects to the change in intensity of continuous illumination from the first intensity to the subsequent intensity, respectively, to give fit information;
wherein the first profile showing a better fit with the control reference profiles compared with the subsequent profile is indicative that the medical condition in the subject is deteriorating from the first time point to the subsequent time point;
or
wherein the subsequent profile showing a better fit with the control reference profiles compared with the first profile is indicative that the medical condition is improving from the first time point to the subsequent time point.

According to a tenth aspect, the present invention provides a method for comparing the pupillary response of a subject with a medical condition associated with an aberrant pupillary response at a first time point and a subsequent time point, comprising comparing:

a first profile representing the pupillary response of the subject to a gradual change in intensity of a continuous illumination of light from a first intensity to a subsequent intensity at a succession of times over a predefined time period at the first time point, and
a subsequent profile representing the pupillary response of the subject to a gradual change in intensity of a continuous illumination of light from a first intensity to a subsequent intensity at a succession of times over a predefined time period at a subsequent time point; with
control reference profiles representing separate pupillary responses of one or both eyes of each subject in a population of control subjects to the change in intensity of continuous illumination from the first intensity to the subsequent intensity, respectively, to give fit information;
wherein the first profile showing a better fit with the control reference profiles compared with the subsequent profile is indicative that the medical condition in the subject is deteriorating from the first time point to the subsequent time point;
or
wherein the subsequent profile showing a better fit with the control reference profiles compared with the first profile is indicative that the medical condition is improving from the first time point to the subsequent time point.

It will be appreciated that a number of subsequent profiles may be obtained for the subject at a number of subsequent time points. For example, a second profile may be obtained at a second time point and a third profile of the subject may be obtained at a third time point. The first, second and third profiles may be compared with each other and with the control reference profiles.

The subsequent profiles may be compared as desired.

From such a comparison, a subject may show an improvement in the medical condition from the first to the second time point and also from the second to the third time point.

From such a comparison, a subject may show a deterioration in the medical condition from the first to the second time point and also from the second to the third time point.

From such a comparison, a subject may show an improvement in the medical condition from the first to the second time point and a deterioration in the medical condition from the second to the third time point.

From such a comparison, a subject may show a deterioration in the medical condition from the first to the second time point and an improvement in the medical condition from the second to the third time point.

It will be appreciated that any appropriate number of profiles of the subject at any appropriate number of subsequent time points may be obtained and compared to assess and/or monitor the subject.

The present invention may also be useful in comparing pupillary responses at different stages of a medical condition associated with an aberrant pupillary response. It will be appreciated that the third aspect of the present invention, for example, may be used to determine that an aberrant pupillary response is associated with the medical condition.

According to an eleventh aspect, the present invention provides a method of comparing a first population of subjects at a first stage of a medical condition associated with an aberrant pupillary response and a second population of subjects at a more severe stage of the medical condition compared to the first population of subjects, comprising

obtaining a first set of reference profiles representing separate pupillary responses of one or both eyes of each subject in the first population of subjects to a gradual change in intensity of a continuous illumination of light from a first intensity to a subsequent intensity at a succession of times over a predefined time period;
obtaining a second set of reference profiles representing separate pupillary responses of one or both eyes of each subject in the second population of subjects to a gradual change in intensity of a continuous illumination of light from a first intensity to a subsequent intensity at a succession of times over a predefined time period;
comparing the first and second sets of reference profiles with control reference profiles representing separate pupillary responses of one or both eyes of each subject in a population of control subjects to the change in intensity of continuous illumination from the first intensity to the subsequent intensity, respectively, to give fit information;
wherein the first set of reference profiles at a first a stage of the medical condition showing less deviation from the control reference profiles compared to the second reference profiles at a more severe stage of the medical condition is indicative that the pupillary response becomes more aberrant with the severity of the condition.

It will be appreciated that the pupillary response of additional populations of subjects at various stages of the medical condition may be compared to identify a correlation between the pupillary response and the severity of the medical condition. Accordingly, the pupillary response may be used to assess the severity of medical condition.

According to a twelfth aspect, the present invention provides a method of comparing a first population of subjects at a first stage of a medical condition associated with an aberrant pupillary response and a second population of subjects at a more severe stage of the medical condition compared to the first population of subjects, comprising comparing:

a first set of reference profiles representing separate pupillary responses of one or both eyes of each subject in the first population of subjects to a gradual change in intensity of a continuous illumination of light from a first intensity to a subsequent intensity at a succession of times over a predefined time period, and
a second set of reference profiles representing separate pupillary responses of one or both eyes of each subject in the second population of subjects to a gradual change in intensity of a continuous illumination of light from a first intensity to a subsequent intensity at a succession of times over a predefined time period; with
control reference profiles representing separate pupillary responses of one or both eyes of each subject in a population of control subjects to the change in intensity of continuous illumination from the first intensity to the subsequent intensity, respectively, to give fit information;
wherein the first set of reference profiles at a first a stage of the medical condition showing less deviation from the control reference profiles compared to the second reference profiles at a more severe stage of the medical condition is indicative that the pupillary response becomes more aberrant with the severity of the condition.

According to a thirteenth aspect, the present invention provides a method of assessing a subject with a medical condition associated with an aberrant pupillary response comprising:

obtaining a profile representing the pupillary response of the subject to a gradual change in intensity of a continuous illumination of light from a first intensity to a subsequent intensity at a succession of times over a predefined time period;
comparing the profile of the subject with a first set of reference profiles representing separate pupillary responses of one or both eyes of each subject in a population of subjects at a first stage of a medical condition, and, one or more other sets of reference profiles, each other set of reference profile representing separate pupillary responses of one or both eyes of each subject in a population of subjects corresponding to a different stage of the medical condition; and
determining the subject as likely to be at a stage of the medical condition corresponding to the set of reference profiles having the best fit compared to the other reference profiles to the profile of the subject.

According to a fourteenth aspect, the present invention provides a method of assessing a subject with a medical condition associated with an aberrant pupillary response comprising:

comparing a profile representing the pupillary response of the subject to a gradual change in intensity of a continuous illumination of light from a first intensity to a subsequent intensity at a succession of times over a predefined time period with:
a first set of reference profiles representing separate pupillary responses of one or both eyes of each subject in a population of subjects at a first stage of a medical condition, and,
one or more other sets of reference profiles, each other set of reference profile representing separate pupillary responses of one or both eyes of each subject in a population of subjects corresponding to a different stage of the medical condition, and
determining the subject as likely to be at a stage of the medical condition corresponding to the set of reference profiles having the best fit compared to the other reference profiles to the profile of the subject.

In particular, the set of reference profiles having the best fit to the profile of the subject may be regarded as the set of reference profiles having the most significant fit with the profile of the subject compared to the other reference profiles.

It will be appreciated that the appropriate number of other sets of reference profiles, each representing a different stage of the medical condition, depends on the medical condition.

The first stage of the medical condition may be an early stage or mild stage of the medical condition. The other stages of the medical condition would differ from the first stage of the medical condition and from each other in terms of presentations, symptoms, severity. In general, the different stages are stages of the medical condition acknowledged in clinical practice and determined based on typical diagnostic criteria for that stage, and the number of stages of the medical condition depends on the medical condition. The present pupillometry method may help to identify the stage of the medical condition of a subject.

For any or all of the seventh to fourteenth aspect of the invention, the method may be performed on stored profiles, reference profiles of a population of subjects and/or control reference profiles retrieved from a tangible computer recordable/readable medium.

It will be appreciated that the real-time images of the eye may be transferred to a separate computer processor which takes and records measurements of the parameter associated with the pupillary response from each of the images. The separate computer processor may also be configured to generate the profile of the subject, profiles of a population of subjects, reference profiles of a population of subjects with a medical condition and control reference profiles,

The profile of a subject, the profiles of a population of subjects, reference profiles of a population of subjects with a medical condition and control reference profiles may also be transferred to the separate computer processor.

The separate computer processor may also be configured to compare and analyse the profile of a subject, reference profiles of subjects with a medical condition and control reference profiles, profiles of a subject before and after administration of a treatment for a medical condition associated an aberrant pupillary response, determine fit and deviation utilising a suitable algorithm, determine normal or aberrant pupillary response using a suitable algorithm, predict if a subject is at risk or suffering from a medical condition using a suitable algorithm, and/or determine if a subject is responding to a treatment for a medical condition associated with an aberrant pupillary response using a suitable algorithm. The separate computer processor is an independent unit from the system of the invention but may be connected to the system via a server.

The present invention also includes a computer processor configured to perform the method according to any aspect of the invention.

The profile may be a plot of the pupillary response with respect to time or a plot of pupillary response with respect to intensity (e.g. a dose response curve). A parameter associated with the pupillary response may be a change in the size of the pupil. Accordingly, the measured parameter may be the diameter of the pupil. Examples of other parameters that reflect the pupillary response include pupil area, rate of change of pupil diameter or light intensity required for a given pupil response, the amount of time required for the pupil to respond by a predefined amount. These other parameters may be derived from the diameter of the pupil. The present invention is not intended to be limited to one particular parameter.

The method according to any aspect of the invention may be performed utilising at least one computer processor. Accordingly, the method according to any aspect of the invention may be implemented using a computer.

The at least one computer processor is configured to carry out instructions to control the light intensity, the filters or mirrors or other reflective surface to direct the light to a selected target region of the retina and optionally light filters to produce the monochromatic light, control the measuring device, generate the profile representing the pupillary response of the eye to the change in intensity of continuous illumination of light corresponding to the succession of times over the predefined time period and analyse and compare the profile of a subject, reference profiles of subjects with a medical condition and control reference profiles, profiles of a subject before and after administration of a treatment for a medical condition associated an aberrant pupillary response, determine fit and deviation utilising a suitable algorithm, predict if a subject is at risk or suffering from a medical condition using a suitable algorithm, and/or determine if a subject is responding to a treatment for a medical condition associated with an aberrant pupillary response using a suitable algorithm.

The method according to any aspect of the invention further comprises storing the images, measured parameters, the profile, profiles, reference profiles, profiles, control reference profiles, fit information, whether an aberrant pupillary response is associated with a medical condition, information as to whether a subject is at risk or suffering from a medical condition and/or information as to whether a subject is responding to a treatment for a medical condition associated with an aberrant pupillary response, information as to pupillary responses at different stages of a medical condition and/or information as to the stage of a medical condition in a subject based on the pupillary response of the subject, as applicable, in a tangible computer recordable/readable medium.

The images, measured parameters, profile, profiles, reference profiles, control reference profiles, fit information, whether an aberrant pupillary response is associated with a medical condition, information as to whether a subject is at risk or suffering from a medical condition and/or whether a subject is responding to a treatment for a medical condition associated with an aberrant pupillary response, information as to pupillary responses at different stages of a medical condition and/or information as to the stage of a medical condition in a subject based on the pupillary response of the subject, as applicable, may be stored in a tangible computer/readable medium.

The present invention accordingly includes a tangible computer recordable/readable medium comprising the images, measured parameters, profile, profiles, reference profiles, control reference profiles, fit information, whether an aberrant pupillary response is associated with a medical condition, information as to whether a subject is at risk or suffering from a medical condition whether a subject is responding to a treatment for a medical condition associated with an aberrant pupillary response, information as to pupillary responses at different stages of a medical condition and/or information as to the stage of a medical condition in a subject based on the pupillary response of the subject, as applicable.

The invention also includes a tangible computer recordable/readable medium comprising a computer program product comprising instructions to cause a computer processor to execute the method according to any aspect of the invention. These instructions are typically written in computer code. These instructions are executed by one or more computer processors for example, to control the continuous light source, change in intensity of the light source, the infra-red light, the camera to capture and/or record images, to measure the parameters from the images, generate the profile of a subject, profiles of a population of subjects, reference profiles of subjects with a medical condition and control reference profiles, compare and analyse the profile of a subject, reference profiles of subjects with a medical condition and control reference profiles, profiles of a subject before and after administration of a treatment for a medical condition associated an aberrant pupillary response, determine fit and deviation utilising a suitable algorithm, determine normal or aberrant pupillary response using a suitable algorithm, predict if a subject is at risk or suffering from a medical condition using a suitable algorithm, and/or determine if a subject is responding to a treatment for a medical condition associated with an aberrant pupillary response using a suitable algorithm. Accordingly, the system according to any aspect of the present invention additionally comprises at least one computer processor configured to control the light intensity, the filters or mirrors or other reflective surface to direct the light to a selected target region of the retina and optionally light filters to produce the monochromatic light, control the measuring device, generate the profile representing the pupillary response of the eye to the change in intensity of continuous illumination of light corresponding to the succession of times over the predefined time period and analyse and compare the profile of a subject, reference profiles of subjects with a medical condition and control reference profiles, profiles of a subject before and after administration of a treatment for a medical condition associated an aberrant pupillary response, determine fit and deviation utilising a suitable algorithm, predict if a subject is at risk or suffering from a medical condition using a suitable algorithm, and/or determine if a subject is responding to a treatment for a medical condition associated with an aberrant pupillary response using a suitable algorithm.

In the present invention, pupillary responses to different wavelengths and intensities of light are used to assess ocular dysfunction. The intrinsically photosensitive retinal ganglion cells (ipRGCs) and visual photoreceptors (rods and cones) differ in their response properties. The ipRGCs are preferentially sensitive to blue light, respond sluggishly to the onset and offset of a light stimulus, and are less sensitive to light than rods and cones. By comparison, rods are most sensitive to bluish-green light, and the cone photoreceptors that mediate color vision are most sensitive to longer-wavelength light (in the green to red portion of the visual spectrum). In the present invention, the wavelength and irradiance of light exposure are manipulated to preferentially stimulate the rods, cones, or the ipRGC response, which provides a window onto the health status of each photoreceptor cell type. For example, blue light can be used to activate rods preferentially at low intensities and ipRGCs at high irradiances, whereas red light can be used to target preferentially the cones. In our approach, we exploit the different response properties of visual photoreceptors and ipRGCs to detect photoreceptor dysfunction associated with different types of retinal diseases. In contrast to prior art, which has relied upon short pulses of light, our invention allows for rapid determination of the dose-response function of the pupillary light reflex. The approach of using a gradually increasing or decreasing light stimulus, as described in the present invention, should perform better at detecting subtle ocular abnormalities that might go undetected using a single light intensity or a small number of steps.

Having now generally described the invention, the same will be more readily understood through reference to the following examples, which are provided by way of illustration, and are not intended to limit the present invention.

### EXAMPLES

### Example 1

Reduced Pupillary Responses to High-Irradiance Blue Light Associate with Visual Field Loss and Optic Disc Cupping in Glaucoma

### Summary

### Design:

Cross-sectional study.

### Participants:

Forty patients with glaucoma ranging from mild to severe, and 161 control patients without ocular disease aged 50 years and older.

### Methods:

Patients were exposed to a 2-minute gradually increasing blue light (469 nm) or red light (631 nm) stimulus designed to sequentially activate rods, cones, and the intrinsic light response of melanopsin-containing retinal ganglion cells. Light was delivered to one eye using a modified Ganzfeld dome with LEDs as the light source. Pupil diameter was recorded using an infrared pupillography system.

### Main Outcome Measures:

Pupillary constriction responses to blue light and red light were compared between controls and patients with glaucoma by constructing dose-response curves across a wide range of corneal irradiances (7 to 14 log photons cm⁻² s⁻¹). In patients with glaucoma, pupillary responses were evaluated relative to standard diagnostic measures including standard automated perimetry (Humphrey Visual Field, HVF) testing and scanning laser ophthalmoscopy measures (Heidelberg Retinal Tomography, HRT).

### Results:

In both healthy controls and glaucomatous eyes, pupillary constriction was greater in response to blue light versus red light. However, pupillary constriction became increasingly impaired in patients with glaucoma as the irradiance was increased beyond 11.5 log photons cm⁻² s⁻¹. Pupillary responses to high-irradiance blue light were associated with severity of the disease, as evidenced by a significant linear correlation between pupillary constriction and HVF mean deviation (r=0.44, p=0.005) as well as HRT linear cup disc ratio (r=0.61, p=<0.001) and several other optic nerve head parameters.

### Conclusions:

The reduced pupillary response to high-irradiance blue light in glaucomatous eyes indicates loss of function of melanopsin-containing retinal ganglion cells. These studies establish that a short-duration light exposure protocol coupled with pupillometry may be used to screen for patients with damage to retinal ganglion cells and/or their efferent projections.

### Details of the Study

### Details of the Methods

### Subjects

Normal subjects were selected from a group of 300 individuals aged 50 years and older who underwent a comprehensive ophthalmic examination at a community polyclinic. The eye examination consisted of tests for visual acuity, Goldmann applanation tonometry to measure intraocular pressure, and automated refraction to assess refractive error. Patients also underwent a slit-lamp examination and examination by a study ophthalmologist.

Forty-eight (48) individuals with primary open-angle glaucoma were recruited from glaucoma clinics at the Singapore National Eye Centre (SNEC). Patients with primary open angle glaucoma (POAG) were defined by the following criteria: the presence of glaucomatous optic neuropathy (defined as a loss of neuroretinal rim with a vertical cup: disc ratio of >0.7 or an inter-eye asymmetry of >0.2, and/or notching attributable to glaucoma) with compatible visual field loss, open angles on gonioscopy, and absence of secondary causes of glaucomatous optic neuropathy. In addition to ophthalmic tests described above, patients underwent standard automated perimetry (Humphrey visual field [HVF] analyzer II model 750, Carl Zeiss Meditec, Dublin, Calif.) and Heidelberg Retinal Tomography (HRT 3, Heidelberg Engineering, Heidelberg, Germany), performed either on the day of chromatic pupillometry testing or within the preceding 3 months. HVF testing was performed with near refractive correction using the 24-2 Swedish Interactive Thresholding Algorithm (SITA) with stimulus size III. Repeat testing was performed if false positive or false negative responses exceeded 33%, or if the fixation loss rate was greater than 20%. Subjects who could not achieve these reliability criteria were excluded. The presence of a glaucomatous hemifield test result outside normal limits and the presence of at least 3 contiguous, non-edge test points within the same hemifield, on the pattern deviation probability plot at P<5%, with at least 1 point P<1%, excluding points directly above and below the blind spot, was defined as glaucomatous. Subjects with mean deviation (MD)≧−6 dB were classified as having mild VF loss; MD between −6 dB and −12 dB as moderate; and MD≦−12 dB as severe VF loss. For HRT, global and segmental disc and cup areas were analyzed using the standard reference plane. Patients who had previously undergone intraocular surgery were excluded. Additional exclusionary criteria included significant nuclear sclerosis greater than grade 2 severity on slit lamp examination, severe retinal or ocular co-morbid conditions including but not limited to diabetic retinopathy and age related macular degeneration, and clinically significant pupillary abnormalities except for relative afferent pupillary defects. Demographic information was collected using interviewer-administered questionnaires. The study was approved by the SingHealth Centralized Institutional Review Board, and all participants provided written informed consent. Research procedures adhered to ethical principles outlined in the Declaration of Helsinki.

### Chromatic Pupillometry

Prior to each light exposure, participants were seated with their head position fixed by a chinrest for at least 1 minute in a dark environment. Light was then administered using a modified Ganzfeld dome, with one eye covered by an eye patch. Patients were exposed to a blue light stimulus (469 nm) or red light stimulus (631 nm) with the order of exposure randomized and counterbalanced. Narrow bandwidth light was provided using light-emitting diodes (LEDs, Nichia Corporation, Tokushima, Japan) that were controlled using a function generator (Keithley Instruments Inc., Cleveland, Ohio). Current was applied to the LEDs over a 2-minute period using a logarithmically increasing function in 14,000 steps. Hence, the light stimulus was perceived as increasing gradually in intensity over time, ranging from 6.8 log photons cm⁻² s⁻¹ to 13.8 log photons cm⁻² s⁻¹ at the level of the cornea. Light levels were calibrated using a portable radiometer (ILT1700, International Light Technologies, Peabody, Mass.) with the sensor placed at the level of the patient's eyes during light exposure. The light exposure was followed by a 1-minute period of darkness, during which the patient remained seated with his/her head in the dome. This was followed immediately by the next light exposure sequence for the other colour of light (1 minute of darkness, 2 minutes of light, and 1 minute of darkness). In the event that a quality eye image could not be obtained for one of the trials, participants underwent a third light exposure sequence (7 out of 300 patients). During each 4-minute light exposure sequence, patients wore a head-mounted eye tracking device that recorded monocular pupil diameter at a rate of 120 samples per second (ISCAN Inc., Woburn, Mass.).

### Data Analysis and Statistics

For each light exposure, pupil diameter measurements were processed to remove blink artefacts in the recording, and then expressed as a percentage of the median pupil diameter during the preceding dark period. Data were reduced by taking the median pupillary constriction response in 0.5 log unit bins from 7 to 14 log photons cm⁻² s⁻¹, resulting in 14 data points per light exposure sequence

$$y = {y}_{0} + \frac{a}{1 + {( \frac{x}{{x}_{0}} )}^{b}}$$

For each patient group the interaction of wavelength and irradiance on the pupillary light reflex (PLR) was examined using two-way repeated measures ANOVA. Similarly, the interaction of ocular health (controls versus glaucomatous eyes) and irradiance on pupillary constriction for each colour of light was examined using two-way repeated measures ANOVA. For those comparisons in which the omnibus test reached statistical significance, pairwise multiple comparison procedures were performed using the Holm-Sidak method. Differences in age between controls and patients with glaucoma were assessed using an unpaired Student's t test assuming unequal variance. In patients with glaucoma, the strength of the linear relationship between pupillary constriction and clinical measures (e.g., HVF mean deviation and linear optic cup disc ratio) was assessed using Pearson's correlation analysis. For all statistical tests, the threshold for significance was set at α=0.05. Statistics were performed using Sigmaplot (Systat Software, Inc., San Jose, Calif.) and SPSS software (IBM Corp., Armonk, N.Y.).

### Results

### Patient Characteristics

Out of the 300 patients who were studied in the polyclinic, 86 individuals were excluded from the analysis due to diagnosis of an ophthalmic condition (e.g., severe cataracts, primary angle closure glaucoma, pterygium, etc.), or were referred to a specialist for a follow-up assessment. In the remaining 214 patients with normal-for-age ocular health, an additional 43 participants were excluded based on the criterion that the baseline pupil diameter measured in darkness differed between light exposure trials by greater than 10%. Technical problems resulted in loss of data in 10 individuals. Therefore, a total of 161 patients were included in the control group, including 107 individuals with mild cataracts, as some clouding of the lens is normal for this age group. Pupillary responses in these subjects were similar to those observed in individuals without cataracts (F_{1,13}<2.30, p>0.13 for main effect of group for both colours of light). Out of the 48 patients recruited from the glaucoma clinic, baseline pupil diameter was not stable in seven (7) individuals, and data loss occurred during the pupillometry recording in one (1) participant. Therefore, data from 40 glaucoma patients was included in these analyses, including persons with mild (n=19), moderate (n=10), and severe (n=11) stage glaucoma. Although the age range was similar between groups, patients with glaucoma were four (4) years older on average (t=3.6, p<0.001) and included a greater proportion of males and ethnic-Chinese individuals relative to controls (Table 1).

TABLE 1
Patient characteristics.

| | | Males | Chinese | Age (mean ± |
|---|---|---|---|---|
| Patient group | N | (no.) | (no.) | SD) |
| Controls | 161 | 55 | 151 | 59.8 ± 6.2 |
| No cataract | 54 | 36 | 49 | 56.4 ± 5.1 |
| Mild/moderate | 107 | 19 | 102 | 61.5 ± 6.0 |
| cataract | | | | |
| Glaucoma | 40 | 22 | 35 | 63.8 ± 6.1 |
| Mild stage | 19 | 9 | 16 | 62.3 ± 6.3 |
| Moderate stage | 10 | 4 | 10 | 64.9 ± 6.1 |
| Severe stage | 11 | 9 | 9 | 65.5 ± 5.6 |

Chromatic pupillometry in patients with normal ocular health

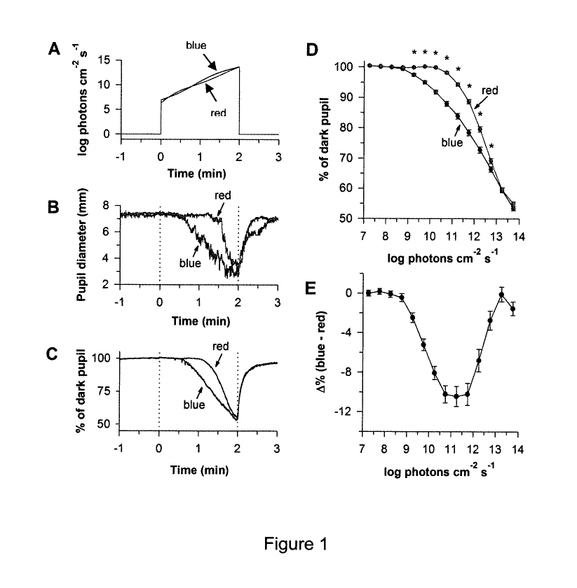

In response to the gradually increasing light stimulus (FIG. 1A), the onset of pupillary constriction occurred much earlier for blue light relative to red light (FIG. 1B-C). Pupil diameter closely tracked the change in irradiance over time for both exposures, followed by a similar time course of re-dilation after light offset. Pupillary responses measured over time were then converted to dose-response curves by grouping results in 0.5 log unit bins (FIG. 1D). The threshold for pupillary constriction was between 9-9.5 log photons cm⁻² s⁻¹ in response to blue light, as compared to 10.5-11 log photons cm⁻² s⁻¹ for exposure to red light. Pupillary constriction responses were significantly greater in response to blue light across wide range of irradiances, from 9-13 log photon cm⁻² s⁻¹ (F_{1,13}=62.18, p<0.001 for interaction; t>3.35 and p<0.05 for all pairwise comparisons), with the greatest difference in spectral responses observed in the middle of this range (FIG. 1E).

Chromatic Pupillometry in Patients with Glaucoma

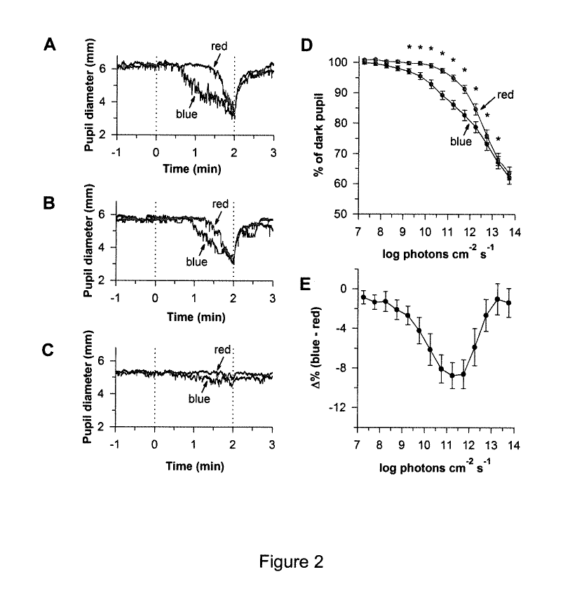

Similar to controls, the onset of pupillary constriction in patients with glaucoma occurred earlier for the blue light stimulus. This was evident across different disease stages of glaucoma, ranging from mild to severe (FIG. 2A-C). As for controls, glaucomatous eyes showed stronger pupillary responses to blue light in the irradiance range from 9-13 log photon cm⁻² s⁻¹ (F_{1,13}=9.89, p<0.001 for interaction; t>2.02 and p<0.05 for all pairwise comparisons) (FIG. 2D), and the greatest difference in pupillary constriction relative to red light was in the middle of this range (FIG. 2E). Overall, dose-response curves to blue light and red light were similar in shape between controls and patients with glaucoma; hence, the wavelength-dependency of pupillary responses was preserved in patients with glaucoma.

### Deficits in Pupillary Responses to High-Irradiance Light in Glaucoma

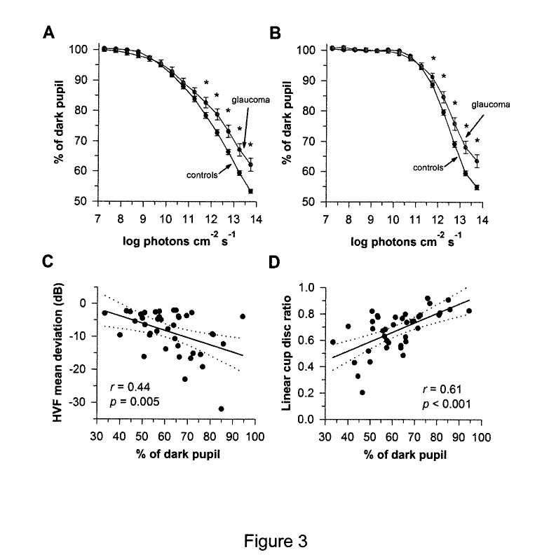

Next, it was evaluated whether the magnitude of pupillary responses to light differed in controls and patients with glaucoma. At lower irradiances, there was no difference between groups in pupillary constriction for blue light and red light stimuli (FIG. 3A-B). As irradiance was increased beyond 11.5 log photon cm⁻² s⁻¹, however, the relative response in patients with glaucoma became increasingly impaired. For both colours of light, there was a significant interaction between group (glaucoma versus controls) and irradiance such that the difference in pupillary constriction between healthy and glaucomatous eyes was greatest at the highest irradiances tested (F_{1,13}>11.38, p<0.001 for omnibus test; t>2.73 and p<0.05 for all pairwise comparisons above 11.5 log photon cm⁻² s⁻¹). Although the age range was similar in controls and patients with glaucoma, the possibility that the difference in pupillary responses between groups might be driven by differences in patient characteristics was considered. Therefore a secondary analysis was conducted in which 40 individuals were selected from the control group who were matched for age (±2 years), sex, ethnicity, and order of light exposure with patients who had glaucoma. Pupillary constriction was impaired at higher irradiances in glaucomatous eyes when these patients were matched with similar control participants (F_{1,13}>5.42, p<0.001 for omnibus test; t>2.28 and p<0.05 for all pairwise comparisons above 12.5 log photon cm⁻² s⁻¹).

Based on these observations whether the pupillary constriction response to high-irradiance blue light (>13.5 log photon cm⁻² s⁻¹) correlated with clinical criteria used for assessing severity of glaucoma, was then tested. In patients with glaucoma, pupil diameter (expressed as a percentage of the dark pupil) exhibited a significant correlation with HVF mean deviation (FIG. 3C; r=−0.44, p=0.005) and linear cup disc ratio assessed by HRT (FIG. 3D; r=0.61, p=<0.001). That is, impaired pupillary responses were associated with greater visual field loss and pathological cupping of the optic disc. Notably, the correlation between the PLR and the linear cup disc ratio was just as strong as the correlation between HVF mean deviation and the linear cup disc ratio (r=−0.59, p=<0.001). By comparison, baseline intraocular pressure (without medication) did not correlate significantly with pupillary constriction, HVF mean deviation, or the linear cup disc ratio (|r|<0.14 and p>0.40 for all comparisons).

Next, the correlation between the magnitude of pupillary constriction at different irradiances with HVF mean deviation and optic nerve head parameters assessed by HRT was systematically evaluated. The strength of the correlation between pupillary constriction and HVF mean deviation increased as the irradiance of light was increased, for both blue light and red light stimuli. A similar relationship was observed for several HRT measures including cup area, cup volume, cup disc ratio, mean retinal nerve fibre layer thickness, and quadrant-specific disc area measurements. The strength of the correlation with optic nerve head parameters was much stronger for pupillary responses to blue light as compared to red light, especially at higher irradiances.

### Example 2

### Differentiating Between Early, Moderate and Severe Glaucoma

Using the chromatic pupillometry methods and analytical approaches described earlier in Example 1 with slight modifications, the pupillary constriction responses to blue and red coloured lights were examined in 29 patients (50 years and older) who presented with early glaucoma, 14 patients with moderate glaucoma, and 12 patients with severe glaucoma (all aged 50 years and above). In this case, the light levels were gradually increased between 8.5 and 14.5 log photon cm⁻² s⁻¹, and subjects were exposed always to blue light first. The pupillary constriction responses in these three groups of patients were compared with the response in a group of 39 control subjects (50 years old and above) exposed to the same light levels.

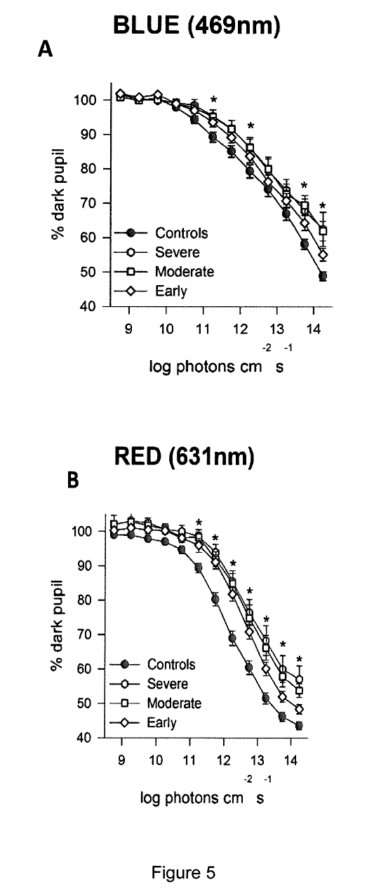

At lower irradiances, there was no difference for pupillary constriction between any of the patient groups for blue light and red light stimuli (FIG. 5A-B). As irradiance of both colours of light was increased beyond 11 log photon cm⁻² s⁻¹, the response in glaucoma patients was increasingly impaired relative to controls. This impairment was seen in all groups of patients, with severe glaucoma showing the maximum change. There was a dose-dependent reduction of pupillary constriction responses with increasing severity of the disease. With the test described here, it was possible to detect even early-stage aberrant responses and differentiate between different stages, including between early glaucoma and the absence of disease in control subjects. Thus, the procedure can be used as a method of assessing disease severity and monitoring progression, as well as detecting persons at risk of developing a more severe condition and who could therefore benefit from a thorough ophthalmic examination.

### Example 3

### Normal Tension Glaucoma (NTG)

The pupillary constriction responses in 26 patients (50 years and older) who presented with normal tension glaucoma were examined as per Example 2. In these patients, the optic nerve and retina are damaged, in a similar way as seen in patients with glaucoma due to increased intra-ocular pressure. However, in normal tension glaucoma (NTG), the normal intra-ocular pressure can make the detection of the condition even more difficult. The disease progress can be slowed or halted, however, with timely medication. Accordingly, the present invention was used to examine the changes in pupillary constriction responses that occur due to optic nerve changes seen with normal intra-ocular pressure.

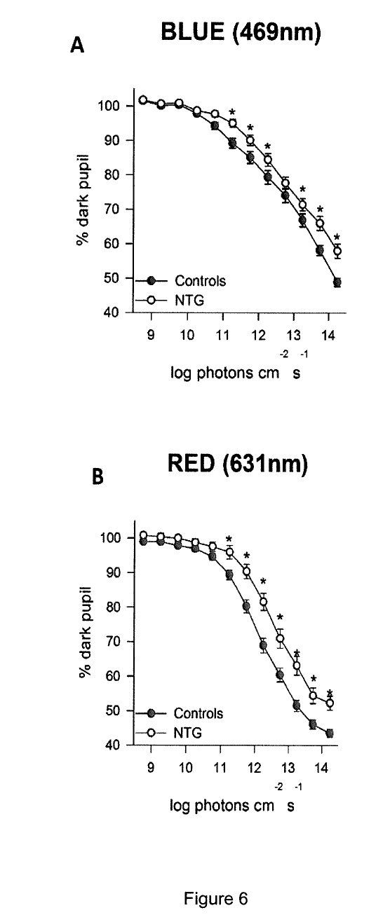

The magnitude of pupillary constriction responses to gradually increasing blue and red lights differed in controls and patients with NTG. At lower irradiances, there was no difference between groups in pupillary constriction for blue light and red light stimuli (FIG. 6A-B). As the irradiance of both colours of light increased above 11 log photon cm⁻² s⁻¹, the response in patients was increasingly impaired relative to controls. Thus, this method can also be used to screen for, monitor and assess patients with normal tension glaucoma.

In the present invention, we describe a novel pupillometry-based method for evaluating ocular abnormalities, and the pupillary responses to progressive increase of light intensity. We have developed and assessed a light stimulus (blue or red), which is increased gradually over time, allowing evaluation of pupillary responses across a wide range of irradiances. The examples demonstrate that a short pupillometry test can be used to detect loss of function of intrinsically photosensitive retinal ganglion cells (ipRGCs) in patients with ocular disease, and that the degree of impairment in the pupillary response correlates with disease severity. In the context of the present invention, these data indicate that the functional integrity of ipRGCs and their efferent projections can be evaluated by measuring pupillary responses to a light stimulus the intensity of which is increased gradually over time.

### Example 4

### Primary Angle Closure Suspects (PACS)

Pupillary constriction responses to blue and red coloured lights were examined in 14 patients (50 years and older) who presented with primary angle closure, using the methods and light levels described in Example 1. The angle between the iris and the cornea was narrow in these subjects. In these cases, the intra-ocular pressure increases due to insufficient drainage of the aqueous humor, and this can lead to optic nerve changes similar to that in primary open angle glaucoma. This ocular problem is easy to treat with timely diagnosis; otherwise, the patient can face loss of vision.

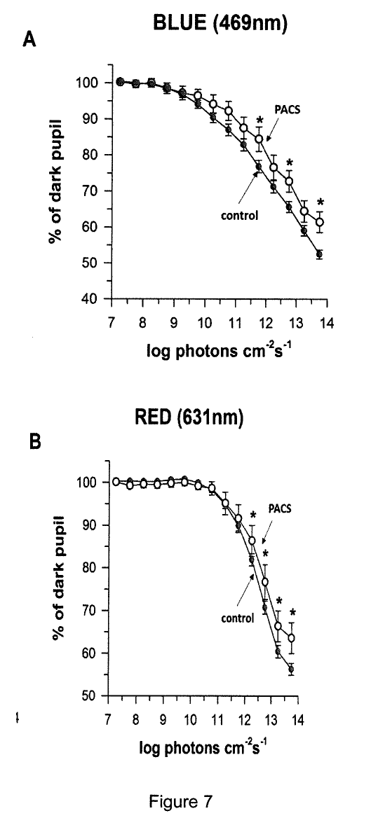

Whether or not there were differences in the magnitude of pupillary constriction responses to blue and red light, between control subjects (n=54, aged 50 years or older) and patients with narrow irido-corneal angles as described above, was evaluated. At lower irradiances, there was no difference between groups in pupillary constriction to blue light and red light stimuli (FIG. 7A-B). As irradiance of blue and red light was increased beyond 11.5 log photon cm⁻² s⁻¹, the response in patients with primary angle closure was decreased relative to controls. Therefore, the invention as described herein can similarly be used to detect aberrant changes in pupillary constriction associated with PACS.

### Example 5

### Optic Neuritis

The method of the present invention was used to explore pupillary responses to light in a 65-year old subject with unilateral idiopathic optic neuritis. Optic neuritis is an inflammatory condition affecting the optic nerve, resulting in sub-acute loss of vision. During an attack, there is profound visual loss and no detectable pupillary response in the affected eye. Using the methods of the present invention it was possible to monitor said pupillary response during, and again after, the attack and demonstrate that, after recovery, pupillary light responses returned to normal, demonstrating reversible loss of function of ipRGCs in optic neuritis. Accordingly, the present invention was used to monitor progression of and recovery from an ocular dysfunction

### Case Report

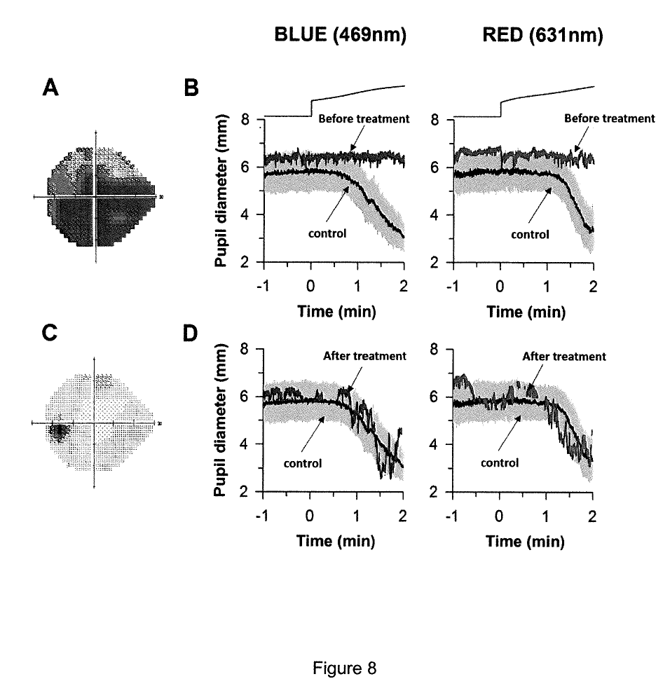

A 65-year-old subject with a past medical history of idiopathic optic neuritis affecting the right eye 2 months earlier, but with full clinical recovery, presented with unilateral severe, acute, painful visual loss in her left eye. Ophthalmic examination disclosed decreased vision to “counting fingers at 3 feet” in the left eye, severe left dyschromatopsia, and a severe left relative afferent pupillary defect; the remainder of the ophthalmic and neurological examination was normal, except mild temporal pallor of the optic disc on the right side. Standard automated perimetry using a Humphrey Visual Field analyzer II (Model 750, Carl Zeiss Meditec, Dublin, Calif.) disclosed diffuse field loss in the left eye (FIG. 8A). The patient's brain MRI scan revealed inflammation within the intraorbital segment of the left optic nerve without any evidence of compression. A systemic work-up did not disclose any abnormality suggesting an underlying infection or inflammation.

The patient underwent chromatic pupillometry testing, during which she was seated in darkness for 1 minute, followed by monocular exposure to blue light (469 nm) or red light (631 nm) provided by light-emitting diodes. The light stimulus was delivered to the left eye using a Ganzfeld dome while the other eye was covered. The light stimulus was increased gradually from 6.8 log photons cm⁻² s⁻¹ to 13.8 log photons cm⁻² s⁻¹ measured at the cornea, and pupil diameter was recorded using an eye-tracking system (ISCAN Inc., Woburn, Mass.). The patient's pupil in the affected eye failed to respond to light (FIG. 8B), even at the highest irradiances tested, suggesting complete loss of function of the ipRGC pathway that mediates the pupillary light reflex. Intravenous treatment with steroids (5 g over 5 days) as described in Optic Neuritis Study Group (2008) resulted in rapid and complete recovery of visual acuity and significant improvement of the visual field in the affected eye (FIG. 8C). In parallel with the patient's recovery of vision, pupillary light responses were comparable with those of healthy subjects in the same age group when assessed 2 weeks after the first chromatic pupillometry test (FIG. 8D).

Hence, the present invention may also be used to monitor and assess visual photoreceptor responses, including dysfunctionality as well as recovery, in other ocular disorders, such as is demonstrated herein for optic neuritis.

Besides optic neuritis, intra-ocular neuronal loss of the retinal ganglion cells can occur, even asymptomatically, in various other inflammatory conditions affecting the central nervous system, such as, for example, multiple sclerosis and neuromyelitis optica. This ocular neuronal loss, which can be detected anatomically by expensive ophthalmic methods (optical coherence tomography) can be a predictor for the further general, neurological morbidity of the disease. The pupillometry system and/or method described herein may predict, detect and monitor disease progression and/or response to treatments, even in the absence of visual loss.

### Example 6

### Diabetic Retinopathy (DR)

Gradually increasing blue and red lights, as described previously in Example 1, were used to examine the pupillary constriction responses in two patients who presented with retinal changes due to diabetes, termed diabetic retinopathy. Diabetes-related changes in the blood vessels of the retina lead to leakage of fluid from them, and other changes, ultimately resulting in retinal damage. The condition eventually leads to loss of vision, although the progress can be slowed with timely intervention and treatment. The present invention was used to evaluate if changes in the pupillary constriction response could be used to detect diabetic retinopathy.

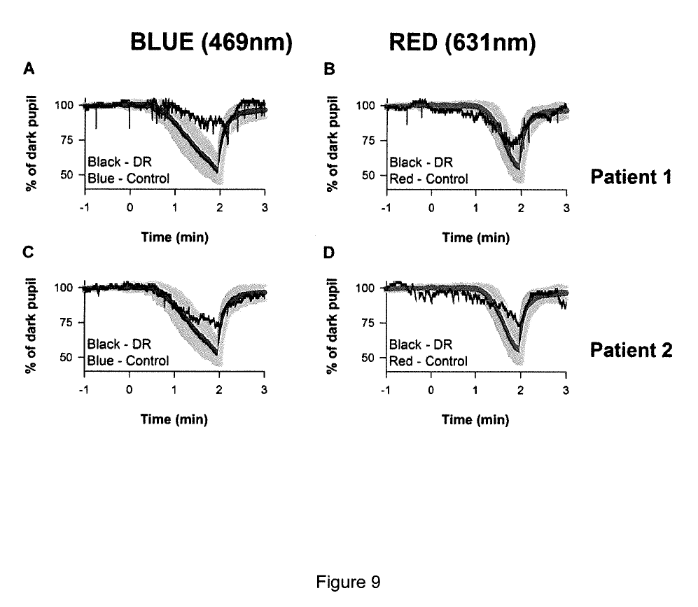

The pupillary constriction responses to blue and red light were qualitatively and quantitatively different from that in 54 control subjects (FIG. 9A-D). The constriction responses in individual patients started late and the constriction was not as strong as that in controls. Thus, this invention was successfully used to detect deficiencies in pupillary response due to diabetic retinopathy.

### Example 7

### Retinitis Pigmentosa

The methods of the current invention, as described in Example 1, were used to explore pupillary constriction responses to blue and red light in a 45-year old male subject with retinitis pigmentosa. Retinitis pigmentosa represents a group of inherited diseases in which there is a gradual loss of vision from an early age leading to degeneration of rods and cones, and ultimately blindness. The patient investigated developed changes in visual acuity before the age of 10, and by the time he came to the clinic, he was blind, unable to detect any light at all. Using chromatic pupillometry, pupillary constriction responses to blue and red light of gradually increasing irradiance were evaluated.

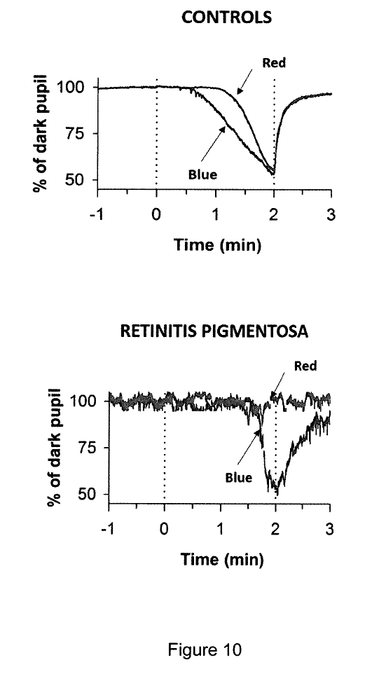

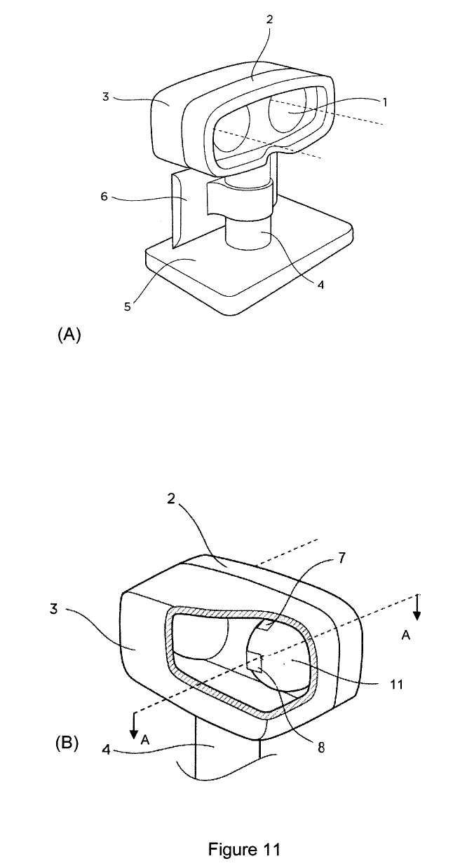

The subject showed no response to red light, when compared to control subjects (n=54, 50 years or older) even at the highest irradiances used (FIG. 10). The patient showed a constriction response to blue light only at the very high irradiances used here. Because rod and cone photoreceptors were not functional in this patient, these results indicate preservation of retinal ganglion cells that mediate pupillary constriction. If used to examine patients from a young age, the methods described herein can help to monitor the progress of this debilitating disease as well as assess responses to treatment (e.g., gene therapy or retinal prostheses).

Using the approach described herein, dose-response curves to light may be rapidly constructed. The ramp-up stimulus that was used allows for examination of rod-cone and melanopsin-dependent RGC activation using a single light exposure sequence and the results indicate that the magnitude of impairment of melanopsin-dependent pupillary responses may be used to infer the degree of damage to RGCs that mediate image-forming vision.

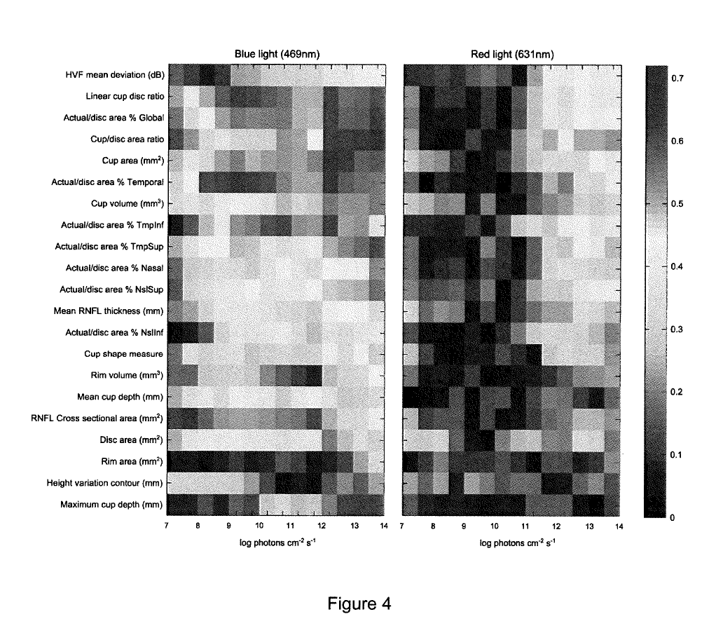

The present Examples describe the examination of pupillary responses across much finer steps in irradiance than used in prior work (Kardon et al., 2009 and 2011), which made it possible to determine the dose-response across a wide range of irradiances, hence providing more detailed information regarding the origin and magnitude of the observed deficit (e.g., see FIGS. 4, 5, 6, 7, 9 and 10). Also, light stimuli was expressed in terms of photon density, which is better for making inferences regarding the contribution of different photoreceptor types to the light response (Lucas et al., 2014).

The study in Example 1 also describes, for the first time, a comprehensive analysis of the relationship between pupillary constriction and numerous optic nerve head parameters. The degree of impairment of pupillary responses to high-irradiance blue light was associated with pathological cupping of the optic disc in glaucoma. Moreover the correlation between these measures was just as strong as the relationship between visual field loss and linear cup disc ratio, indicating that the PLR carries substantial information about the health of RGCs and the optic nerve.

The present Examples describe a chromatic pupillometry method that may be used to assess loss of ipRGC function associated with various ocular disease. In diseases such as the different forms of glaucoma, diabetic retinopathy, retinitis pigmentosa, as well as others such as age-related macular degeneration, optic neuritis, gradual loss of vision may go unnoticed by patients for a long time. Consequently, patients often report to the clinic after occurrence of substantial and irreversible damage affecting the retina and optic nerve. This method facilitates detection of early visual pathways dysfunction.

The method can be used to detect diseases that affect the eye and/or central nervous system, resulting in impaired pupillary light responses. This includes neurodegenerative, inflammatory, and developmental diseases.

The retina and its neurons being easily accessible, this system may also detect and monitor more widespread neuronal loss, occurring in neurodegenerative conditions such as, for example, Alzheimer's disease, other dementias, Parkinson's disease and other extrapyramidal syndromes, or inflammatory conditions such as multiple sclerosis and neuromyelitis optica.

A major advantage of pupillometry-based methods is that they can be adapted for population screening in the form of a portable device, e.g. as a desktop system or, alternatively, incorporated into specialized goggles (Ozeki et al., 2013; Herbst et al., 2011 and Lorenz et al., 2012), that can provide objective data on the integrity of the pathway from retina to the midbrain. Pupillometry systems may also be used to screen for early damage to the visual system in population studies and community samples. This allows for identification of at-risk patients who should undergo a comprehensive ophthalmic examination, in order to treat and/or halt the progression of glaucoma and other ocular diseases.

### Example 8

### Desktop System and Wearable System

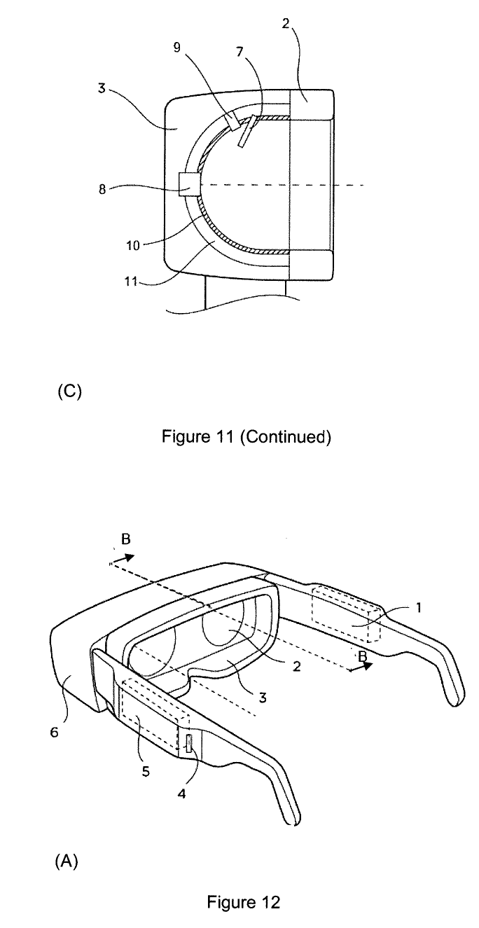

An embodiment of a desktop system is shown in FIG. 11. As shown in FIG. 11(A), the system has a unit comprising two chambers (1), one for each eye of a subject. Each chamber comes with an opening for the respective eye of the subject to look into. If one eye only is being tested, the other opening may be covered in a suitable manner. Alternatively, the continuous light, the infra-red light source and the infra-red camera in the other chamber need not be switched on. There is a casing (3) covering the chambers

The unit is fitted with an eye-piece (2), which may be suitably padded for the comfort of the subject. The system comes with a stand (4) and the base (5). The height of the unit may be adjusted with respect to the stand (4) as appropriate for the subject to look into the chamber(s). The system further comprises a computer processor which may, for example, be located inside the stand (4), for compactness. The system also comprises a display interface (6) to display data.

Referring to FIGS. 11(B) and (c), the source of the continuous light (9) is located in a suitable position within each chamber (1), together with a reflector (7), which may be adjusted to direct the light to the eye. The internal wall of each chamber (10) is also reflective. The outer wall (11) of each chamber is impenetrable to light. The infra-red light source and the infra-red camera (collectively depicted as 8) are also suitably positioned within the chamber (1) to capture the images of the eye in real-time.

The subject looks into one of the chambers and when he is ready, the continuous light source is turned on, the infra-red light source and infra-red camera are also turned on to capture the images in one of the chambers. The computer processor takes measurements, generates a profile, analyses the profile and displays the data on the display interface.

It will be appreciated that in an alternative embodiment, there is only one chamber but two openings, one for each eye. In this alternative embodiment, when one eye is being tested, the other opening may be suitably covered up.

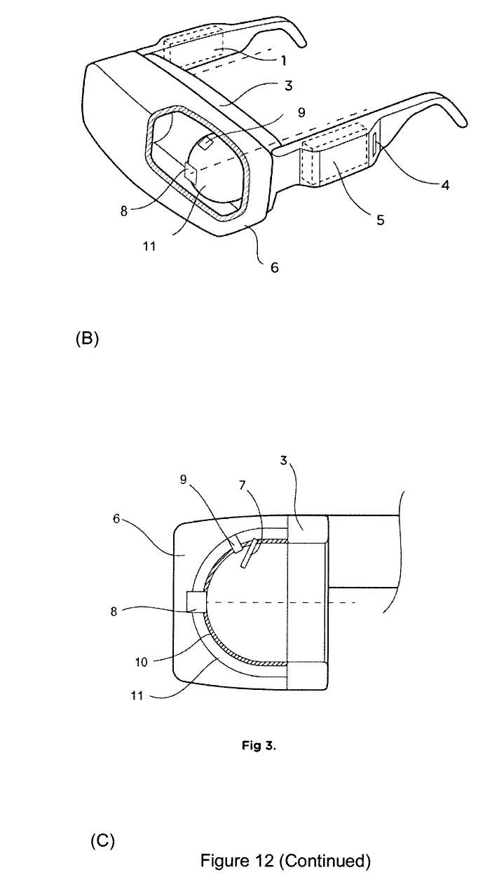

An example of a wearable system in the form of a pair of goggles is shown in FIG. 12. As shown in FIG. 12(A), there are two chambers (2), one for each eye. Similar to the desktop system, each chamber comes with an opening for the respective eye of the subject to look into. If one eye only is being tested, the other opening may be covered in a suitable manner. Alternatively, the continuous light, the infra-red light source and the infra-red camera in the other chamber need not be switched on.

The goggles is fitted with an eye-piece (3), which may be suitably padded for the comfort of the subject. There is a casing (6) which covers the two chambers.

The goggles may be equipped with two computer processors (1, 5) each of which may be located within each earpiece of the goggles, respectively. In the simplest form, each computer processor (1, 5) is typically a microprocessor and only records the images in real-time. Alternatively, there may be only one computer processor which controls the function of both chambers. There may also be a portal (4) for either connecting with another computer processor or retrieving stored data from each computer processor (computer processor 5 for example).

FIG. 12(B) shows a cut-out view of the goggles, and the two chambers can be seen within the casing (6).

Referring to FIG. 12(C), the continuous light source (9) and reflector (7) for directing the continuous light source to the eye are located at an appropriate position in the chamber. The infra-red light source and the infra-red camera (collectively depicted as 8) are also located at an appropriate position in the chamber. The internal wall of the chamber (10) is also reflective. The outer wall (11) of the chamber is impenetrable to light.

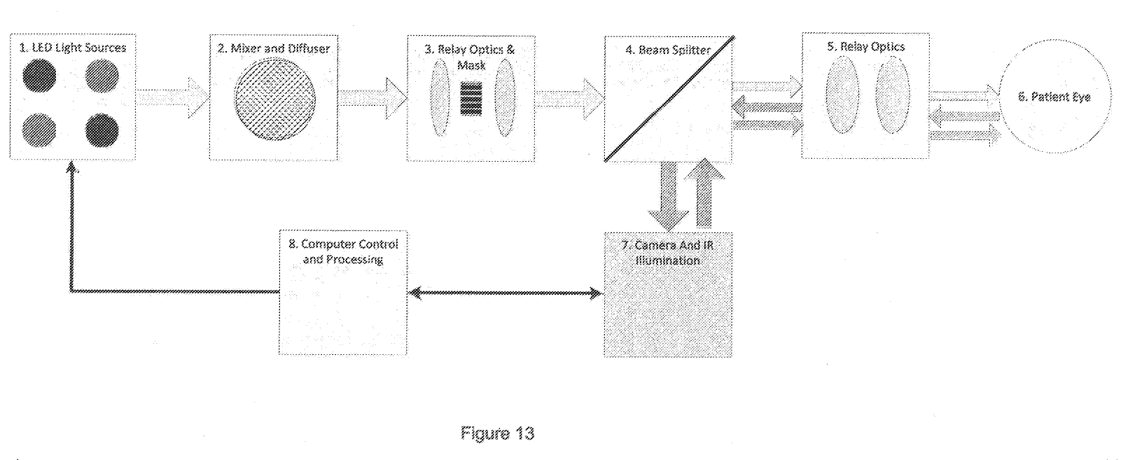

A plan of the components of the system is shown in FIG. 13. It will be appreciated that system includes these components.

### REFERENCES

Chang et al., (2013) Investigative Ophthalmol & Visual Science 54(8):5596-601
Herbst et al., (2011) Front Neurology 2:10
Kardon et al., (2009) Ophthalmology 116(8):1564-1573
Kardon et al., (2011) Ophthalmology 118(2):376-381
Kankipati et al., (2011) Investigative Ophthalmology & Visual Science 52(5):2287-2292
Lorenz et al., (2012) Investigative Ophthalmology & Visual Science 53(9):5641-5642
Lucas et al., (2014) Trends in Neuroscience 37(1):1-9 Optic Neuritis Study Group (2008) Archives of neurology 65(6):727-732
Ortube et al (2013) Investigative Ophthalmology & Visual Science 54(1):9-17 Ozeki et al., (2013) British Journal of Ophthalmology 97(12):1538-42
Tatham et al., (2014) Ophthalmology 121(6):1185-93
U.S. Pat. No. 7,334,895

1. A method for assessing a pupillary response of a first subject to a photic stimulus comprising:
illuminating the first subject's eye with a continuous illumination starting at an intensity and gradually changing the illumination intensity to a subsequent intensity over a predefined period of time;
measuring a parameter associated with a papillary response of the first subject's eye to the change in intensity of the continuous illumination, the measuring being performed-at a succession of times over the period of time; and
obtaining a first profile representing the pupillary response of the first subject's eye to the change in intensity of the continuous illumination corresponding to the succession of times.

2. The method of claim 1 further comprising:
obtaining a second profile representing the pupillary response of the eye to the change in intensity of the continuous illumination corresponding to the succession of times;
comparing the first profile of the first subject's eye with the second profile of the first subject's eye; and
analyzing if there is any deviation between the first profile and the second profile of the first subject's eye.

3-14. (canceled)

15. The method according to claim 1, wherein the continuous illumination is gradually changed from the intensity to the subsequent intensity at a constant rate.

16. The method according claim 1, wherein the continuous illumination is gradually changed from the intensity to the subsequent intensity logarithmically.

17. (canceled)

18. The method according claim 1, wherein the continuous illumination is decreased from the intensity to the subsequent intensity.

19. The method according to claim 1, wherein the continuous illumination is directed to the entire retina of the first subject's eye.

20. The method according to claim 1, wherein the continuous illumination is directed to a selected target region of the retina of the first subject's eye.

21. (canceled)

22. The method according to claim 1, wherein the illumination comprises a narrow band of wavelengths.

23. (canceled)

24. The method according to claim 1, wherein the method further comprises exposing the first subject's eye to multiple sequences of a continuous illumination starting at the intensity and gradually changing the intensity of the light to a subsequent intensity over a predefined period of time.

25. The method according to claim 24, wherein each sequence varies from another sequence in terms of at least one of the following characteristics: wavelength, chromaticity, duration, intensity range and the rate of change of intensity.

26-55. (canceled)

56. The method of claim 1 further comprising:
illuminating a second subject's eye with a continuous illumination starting at the first intensity and gradually changing the illumination intensity to a subsequent intensity over a predefined period of time;
measuring a parameter associated with a pupillary response of the second subject's eye to the change in intensity of the continuous illumination, the measuring being is performed at a succession of times over the period of time;
obtaining a third profile representing the pupillary response of the second subject's eye to the change in intensity of the continuous illumination corresponding to the succession of times;
comparing the first profile of the first subject's eye with the third profile of the second subject's eye; and
analyzing if there is any deviation between the first profile of the first subject's eye and the third profile of the second subject's eye.

57. An apparatus for assessing a pupillary response of a subject to a photic stimulus, the apparatus comprising:
an image capturing device configured to capture images reflected from the subject's eye in real time;
an illumination source with adjustable light intensity configured to illuminate at least one of the subject's eyes with a continuous illumination starting at an intensity and gradually changing to a subsequent intensity over a predefined period of time:
a measuring device configured to measure a parameter associated with a pupillary response of the subject's eye to the change in illumination intensity of the continuous illumination at a succession of times over the period of time;
a computer processor configured to generate a profile representing the pupillary response of the subject's eye to the change in illumination intensity of the continuous illumination corresponding to the succession of times over the predefined period of time.

58. The apparatus according to claim 57, wherein the continuous illumination is directed to the entire retina of the subject's eye.

59. The apparatus according to claim 57, wherein the continuous illumination is directed to a selected target region of the retina of the subject's eye.

60. The apparatus according to claim 57, wherein the illumination source provides illumination of a narrow band of wavelengths.

61. The apparatus according to claim 57, wherein the apparatus is adapted to present multiple sequences of a continuous illumination of light starting at an intensity and gradually changing the intensity of the light to a subsequent intensity over a predefined period of time.

62. The apparatus according to claim 61, wherein each sequence varies from another sequence in terms of at least one of wavelength, chromaticity, duration, intensity range and the rate of change of intensity.
